(19) United States
(12) Patent Application Publication
(10) Pub. No.: US 2014/0205660 A1
(43) Pub. Date: Jul. 24, 2014

(54) Abscisic acid against cancer

(71) Applicant: Gonzalo Romero M, Palm Harbor, FL (US)

(72) Inventor: Gonzalo Romero M, Palm Harbor, FL (US)

(21) Appl. No.: 13/694,941

(22) Filed: Jan. 22, 2013

(51) Int. Cl. A61K 31/19; A61K 9/48

(52) U.S. Cl. CPC A61K 31/19; A61K 9/4891
USPC 424/456; 514/557

(57) ABSTRACT

Abscisic Acid (ABA) a naturally occurring plant hormone has been identified in this invention with potent properties to fight cancer. ABA is able to produce a hyperpolarization condition on plasma membrane through a decrease of intracellular Na⁺ and K⁺. Such phenomenon is produced in cancer cells by mediation of ion channel and activation of the signaling G-protein pathway. ABA aborting sustained depolarization in malignant tissue will produce a change in the configurational state of cell from damage to a normal state. Additionally, a positive polarization of hCG outer layer accomplished through a removal of electrons will permit immune system cells coming close to cancer cells for destruction. The ABA discovery functioning as a cytokine in human granulocytes and anticancer properties late researches, confirm ABA as the key substance used by immune system cells against cancer. ABA can't be biosynthesized and degraded in a cancer anaerobic metabolism lacking in oxygen.

### CROSS-REFERENCE TO RELATED APPLICATIONS

This application is a continuation in part of patent application Ser. No. 12/655,006 filed on Dec. 22, 2009, which is a continuation in part of patent application Ser. No. 11/472,128 filed on Jun. 21, 2006, which is based upon and entitled to the benefit of the provisional patent application Ser. No. 60/692,617 filed Jun. 22, 2005, the subject matter of which applications is incorporated herein by reference.

### BACKGROUND OF THE INVENTION

1. Field of the Invention

This invention has been found in the field of medical treatments of drugs biologically affecting the human body and related to counteracting different types of cancer, which have potentially unlimited growth and expand locally by invasion and systemically by metastasis.

2. Description of the Prior Arts

Abscisic acid (ABA) is a natural occurring plant hormone also denominated Abscisin II or Dormin It is chemically named as [S-(Z,E)]-5-(1-Hydroxy-2,6,6-trimethyl-4-oxo-2-cyclohexen-1-yl)-3-methyl-2,4-pentadienoic acid (Merck Index 1996, p. 2). ABA molecular structure is showed in FIG. 1. ABA structure is a 15-c sesquiterpene synthesized in chloroplasts and likely also under extra chloroplasts biosynthetic pathway. ABA is a weak acid (C₁₅H₂₀O₄) soluble in many organic solvents. Addicott F. T et al. 1969, Milborrow B. V 1974, Walton D. C 1980, and Zeevart Jan A. D et al. 1988 have studied its physical and chemical properties.

The natural compound has been indicated as (S) or (+), its synthetic substance as (RS) or (+/−), and its racemic enantiomer as (−).

ABA natural form (+), and derivatives or analogs, all of these are subjects of the present invention.

Some of the ABA derivatives or analogs have been defined by Milborrow M. V 1974, pp. 261-272. Many of those ahead mentioned substances have been found by researchers generating a variety of inhibitor effects in plants.

ABA as a potent inhibitor has been studied through an extensive bibliographical reference in biochemical and physiological mechanisms such as: water stress and stomata control, dormancy, abscission, senescence, growth inhibition, and hydraulic conductivity.

In relation to seed dormancy, exogenous ABA prevented precocious germination of immature embryos of several species when cultured in vitro, and, ABA produced this phenomenon during the early stages of embryogenesis (Zeevaart Jan A. D et al. 1988). Abscission which is a lysigenous breakdown of cells accelerated by a rapid development of pectinases, cellulases, and proteases has been suggested by Borman et al. 1967, p. 125, and by Addicot et al. 1969, p. 156. Senescence, identified by multiple effects such as fruit maturation, aging and plant death has been observed by Milborrow 1974, p. 293. In addition, Glinka et al. 1971, 1972 and Windsor et al. 1992, p. 59, have mentioned an increase of membrane permeability caused by ABA. Ethylene has been related to the phenomenon of abscission and fruit maturation.

### Plant Growth Inhibition and Flowering

ABA molecule in cis configuration is an active substance, but the isomerization of the carbon 1, where the carboxyl group is located, can change to a trans configuration position. In acidic conditions, the ABA molecule has a maximum light absorption in the area of the spectrum of the ultraviolet (UV) at 262 nm, with a shoulder at 240 nm. In basic conditions, the maximum occurs at 245 nm and is about 20% more intense (Milborrow B. V 1974). Intense hit of the UV light produces an inactivation of ABA molecule, nevertheless far red light activates ABA and also stimulates its biosynthesis (Tucker D. J 1971, 1972, 1973, 1975, 1976 and 1977). ABA isomerization can also be produced by chemical reactions with solvents (Milborrow B. V 1974).

The ABA action in nature might be observed during the seasons of the year, because this molecule is the most important plant growth inhibitor. During the summer, on the north hemisphere, the UV light is more intense than during the winter. UV light inhibits ABA, and due to this hormone holds the main role as growth inhibitor, the plant development and growth during the summer is maximum. During the winter UV lights decreases, and the dull light (far red light) increases, activating ABA and reducing plant growth. This process, also produces dormancy of seeds and buds, and prepares the plants for the hard and cold winter.

Researchers have confirmed ABA properties as plant growth inhibitor. ABA inhibited oat mesocotyl growth by increasing ABA or dormin concentrations, whatever levels of indol acetic acid (IAA) or gibberellins (GA) were given (Milborrow, 1966, p. 154). ABA also inhibits tomatoes lateral buds outgrowth (Tucker, 1977). Probable changes in tissue growth by ABA effects might be provoked in nucleic acids and protein metabolism (Dekock et al. 1978, p. 508). According to Zeevaart Jan A. D. et al. 1988, page 457, ABA can inhibit root growth as well as promote growth of this organ. ABA root growth contradictory effects are explained, because enlarge of the root system is combined with increased hydraulic conductance and water uptake. The latter effects are considered mechanisms to defend the plant against water stress.

ABA also exhibits a correlation with the plant photoperiod and flowering. In fact, ABA and the pigment protein phytochrome control in plants the photoperiod by reversing the nature of the pigment, according the received amount of red and far red light.

### Stomatal Movements

ABA acts in many physiological processes in plants, especially during stomata closure in conditions of limited water supply. Stomata closure involves loss of ions and reduction of osmotic osmotic pressure (Mansfield et al. 1971, p. 147; Dekock et al. 1978, p. 506; Macrobbie, 1997, p. 515). ABA loss or entry of anions and cations is mediated by signaling activation of ion channel (Schroeder et al. 2001, p. 328; Macrobbie, 1997, p. 515).

As it expresses the former statement, stomata movements are correlated in increasing or decreasing stomata osmotic contents. These osmotic changes, cause water to move in or out from guard cells, causing them to either expand (become turgid) or become dehydrated. The stoma is open when it expands and is closed when it becomes dehydrated. Different thick of the guard cell wall causes stomata to open. The wall, which is adjacent to the pore, is thicker and less elastic and flexible than the external wall. Turgor will produce the external wall to expand more than the internal wall, which is close to the pore.

Apparently, ABA in the apoplast must be inhibited or moved during plant rewatering, which starts to open stomata. Inhibition mechanisms probably comprise: relocation of the hormone in other plant compartments outside the stomata complex, ABA uptake into cytoplasm to be metabolized or stored, and inhibition of synthesis.

In normal conditions, stomata opening and closure respond to light, without exogenous application of ABA. X-ray images in tobacco leaves demonstrate a K⁺ increase in guard cells as stomata open in the light, and a decrease as stomata close in the dark (Sawhney et al. 1969, pp. 1351-1353; Humble et al. 1971, pp. 448-450).

Definitively, ABA is involved in stomata changes during the days and nights. According to Zeevaart Jan A. D. et al. 1988, page 456, the amount of ABA in the apoplast can double upon transfer to darkness or in response to a pH change in the chloroplast stoma during stress. A classical model proposes that, drought stress roots produce ABA, which is transported to the leaves via the xylem. Water stress may happen in roots by soil water deficiency and as well as in leaves caused by humidity conditions. According to Schroeder et al. 2006, page 1024, stress may occur in leaves by lowering the ambient humidity. It produces ABA release in leaves via hydrolysis of a pre-existing pool of inactive glucose-conjugated ABA (ABA-GE) FIG. 77. This inactive form is located in vacuoles, xylem sap, and probably in the cytosol and cell wall as well (Dietz et al. 2000). Lee et al. 2006 demonstrated that, the cleavage of ABA-GE by a specific beta-glucosidase (ATBG1), is a mechanism for ABA release quick response to dehydration and also “day/night” conditions.

During normal stomata opening, uptake of K⁺ is mediated by K⁺ ion pump (Sawhney et al. 1969, p. 1350). Outlaw 1983, in his review “Current Concepts on the Role of Potassium in Stomata Movements”, remarks in the abstract, p. 302, that K⁺ uptake by plant cells is mediated by an ATPase that pumps protons across the plasmalema. In normal cells, cytoplasmic K⁺ has a bigger concentration than K⁺ in the apoplast, thus, it seems obvious a use of energy by cells for transporting ions as K⁺ against a concentration gradient. Loss and entry of ions are mechanisms involved in stomata movements. Due to that, plasma membrane ion transport is closely correlated to cancer cell normalization and apoptosis, it is studied below in detail.

### Loss of Solutes by ABA Effect

Mansfield et al. 1971, p. 147, showed in histochemical tests, that guard cell K⁺ concentration of C. communis was reduced by ABA treatment, while the starch content of chloroplasts increased.

ABA also moves other cations as Ca²⁺ to be transported inward. (MacRobbie 1997, p. 515; Schroeder 2001, p. 328), and anions to be transported outward (Schroeder 2001, p. 328).

Dekock et al. 1978, p. 506, working with Lemna gibba fronds and ABA, detected a marked intracellular decrease in K⁺ and Na⁺, an increase in Ca, Mg, Fe, and insoluble P and a marked decrease in P/Fe and K/Ca ratios.

MacRobbie 1997, page 515, also confirms and gives a better explanation of the phenomenon. ABA closes stomata pores by inducing net loss of K⁺ salts (including rubidium) from guard cells, involving net efflux of both anions and cations from the vacuole to the cytoplasm, as well as from the cytoplasm to the outside. ABA plasma membrane ion transport is produced by the activation of the ion channel and ion pump.

Humble et al. 1971, p. 451, under research of stomata movements determined that K⁺ is the specific ion involved, not showing significant importance, the rest of the ions. It takes relevance, due to that ABA also moves other cations as Ca²⁺, to be transported inward and anions to be transported outward. This investigation defines that, potassium is the most important solute in plants.

ABA also inhibits K⁺ uptake, which is required to prevent stomata opening (Schroeder et al. 2001, p. 328). According to Raschke K 1987 and Shimazaki K et al. 1986, ABA prevents stomata opening by rapidly blocking H⁺ extrusion and K⁺ influx (cited in Zeevaart Jan A. D et al. 1988). ABA in roots has a similar effect as it is exerted in leaves when it prevents stomata opening. In excised barley roots of Hordeum distichon, ABA inhibits accumulation, transport and uptake of K⁺ and Na⁺ to avoid osmotic stress during drought (Behl et al. 1979, p. 335, vol 95, Number 1, International Journal of Plant Physiology). It is also possible to observe Na⁺ participation in stomata control and definitive influence of ABA over cation Na⁺. Wilmeri et al. 1969, also demonstrated that Na⁺ and K⁺ are important in stomata mechanisms.

According to research of Behl et al. 1979, already mentioned, it was found that ABA in barley roots inhibited the transport of both ions to a similar degree. It means that, ABA has no selectivity over K⁺ and Na⁺ under general conditions. Nevertheless, ABA may turn out to affect the uptake of K⁺ and Na⁺ differentially by stimulating the uptake of Na⁺ only. Investigations of E. Blumwald et al. define the capacities of ABA as a salt anti-stress hormone in plants. ABA counteracts salt stress by absorbing sodium and storing this element in vacuoles, and also exporting the ion out of cell.

### ABA Target Cells

Long time ago, it has been discovered that, bits of tissue from almost any part of the plant could be excised and grown in cultures. The new cells are not usually organized as differentiated cells, but they produce a formless mass called callus. It can divide perpetually without any decrease of the growth rate, if it is provided fresh medium time to time. On the plant, such cells may have a finite life span and form differentiated cells such as roots and buds, if the appropriate growth hormones are supplied (Galston A. W, 1994). ABA as plant hormone is directly involved in plant cell differentiation.

In plants, the ABA target cells are embryonic cells, meristems and differentiated cells as stomas. It has been proven ABA action in inhibition of seed and bud germination, and in control of stomata closure.

It has also been defined that, tumors are composed of highly aggressive undifferentiated cells and differentiated as well. Undifferentiated cells are biochemically similar to embryonic cells, because the increased expression of embryonic genes in such cells.

Stomas as cancer cells as well, accumulate solutes and water. In plants, during the stage of stomata opening, stomas store mainly K⁺ and water. On the other hand, animal cancer cell accumulates Na⁺ and water. An apoptotic process makes that, cells suffer a phenomenon of shrinkage, which is a mechanism physiologically similar to the mechanism of ABA stomata closure in plants.

### Positive Polarization and Hyperpolarization Effect in Membrane Cell

Tanada 1972, p. 461, in his study “Antagonism Between Indoleacetic Acid and Abscisic Acid on a Rapid Phytochrome-Mediated Process”, concluded that phytochrome acts in conjunction with hormones such as IAA and/or ABA to bring fast changes in surface charges. Tanada suggested these hormones have opposing effects on the surface potentials: ABA inducing a positive, and IAA inducing a negative potential. ABA plant synthesis location has been recognized in chloroplasts mediated by phytochrome's absorbing light, which increases ABA concentration. Tanada's experiment demonstrated that, red photons could increase concentration of ABA relative to that of IAA, thereby bringing about a positive membrane potential, which causes adhesion of root tips to a negatively-charged glass surface.

Hartung et al. 1980, pp. 257-258, recorded a hyperpolarization effect of ABA in Lemna gibba G1, on a membrane electrochemical potential difference (EPD). Treatment with 10-100 mcm, ABA produced a potential increase in average of 85 mV. This research mentions that, a decreased intracellular K⁺ concentration could generate membrane hyperpolarization of Lemna cells. After 15 hours ABA treatment, EPD increased from −109 mV to −194 mV. K⁺ outside of the cytoplasm causes a positive charge on plasma membrane and it increases charge difference between the cytoplasm and surface membrane.

EPD results, from a separation of positive and negative charges across the cell membrane. This separation of predominantly positive charges, outside and negative charges inside the membrane at rest, is maintained because the lipid bilayer acts as a barrier to the diffusion of ions. It gives rise to an EPD. The resting potential can be disturbed whenever there is a net ion efflux into or out of the cell. A reduction of the charge separation is called depolarization, and an increase in charge separation is called hyperpolarization. In normal cells, a higher concentration of anions relative to K⁺ mainly produces a net negative charge inside of cells. A net positive charge outside of cells is produced by a bigger concentration of Na⁺ relative to Cl⁻. The main negative charge inside of cell is held by proteins, usually named intracellular protein matrix.

The ABA hyperpolarization phenomenon mentioned by Hartung et al. 1980 is distinct to the ABA positive polarization effect researched by Tanada 1972. The latter effect is a subtle phenomenon related intimately to the first one, which it will be disclose widely ahead in this invention.

ABA in Relation to a Physiological Relationship with Mammalians

Le page-Degivry et al. 1986, pp. 1155-1156, reported an ABA presence in the central nervous system of pigs and rats. ABA identification by using a radioimmunoassay, in different tissues, demonstrated a bigger amount of ABA found in brains than any of the other tissues. The final product of purification had the same properties as ABA, inhibiting stomata aperture of abaxial epidermis strips of Setcreasea purpurea boom (Commelinaceae). They remarked that, the ABA presence in brain tissue cannot be considered an ABA containing diet consequence, and suggested metabolic pathways identification for ABA biosynthesis in the brain.

ABA presence has been also reported in lower metazoan (marine sponges and hydroids), responding to environmental stimuli as temperature rise in sponges, and light exposure in hydroids (Zocchi et al. 2001, Puce et al. 2004). In humans, ABA has been found stimulating several functions in immune system cells, such as in: granulocytes, lymphocytes, fibroblasts, mesenchymal stem cells, platelets and monocytes (Bruzzone et al. 2007, Zocchi et al. 2008). Also ABA has been identified as an endogenous stimulator of insulin release in human pancreatic islets (Bruzzone et al. 2008).

### General Plant Hormone Effects in Humans

Naturally occurring cytokinins, such as, kinetin and zeatin, which intervene in plant cellular division have been promoted and patented in Europe and USA, for treatment of human skin aging by an international biopharmaceutical corporation denominated Senetek. These patents have showed and proven that, kinetin is capable of delaying or preventing a host of age-related changes of human skin fibroblasts grown in laboratory culture.

Fibroblasts are believed to be at the center of age-related changes of the skin. These cells produce collagen and elastin, the two proteins most clearly tied to the development of wrinkles, sagging and laxity of the skin. Additionally, GA has been structurally found as molecules roughly analogous to the steroid group of animal hormones. Steroids have an enhancing effect in human cells, and it has been used in sport activities for increasing musculature size. In plants, GA produces cellular elongation, which is a similar enhancing effect induced by steroids in humans.

### ABA in Relation to the Disease of Cancer

This invention was initially started by considering that, plant hormones such as IAA, GA and cytokinins stimulate cellular growth in plants. Conversely, ABA manifests an antagonist effect in plants by producing cellular growth inhibition.

ABA was mentioned for the first time in relation to cancer, by Dr. Virginia Livingston in her U.S. Pat. No. 3,958,025 (1976) denominated, “Abscisic Acid Tablets and Process”. In this invention, she experimented with ABA in mice proving ABA neutralizing properties of a Microbic Chorionic Gonadotropin, which was similar to the Human Chorionic Gonadotropin (hCG).

Such statement, defined by Dr. Livingston was essentially a complementary or collateral research, beside her central focus of investigation, which was about a type of tumor-associated bacteria named Progenitor cryptocides. According to Dr. Livingston, such bacterium expresses hCG, which turns it out in developing cancer. Such findings reported back in the early 1970's were erroneously dismissed. After Dr. Livingston's research, it was recognized that, others types of tumor-associated bacteria also express this hormone and cancer.

Some coagulase-negative Staphylococcus species isolated from cancer patients expressed hCG, but not every isolated bacterial strain did it, H. Acevedo (1985), p. 860. Such studies confirmed that, Progenitor cryptocides is not the unique bacterium expressing hCG as postulated by Livingston and Associates, but her conclusions and investigations were generally right. Dr. Livingston's findings were a first indication of a possible synthesis of a mammalian protein hormone by a microbial organism, Cohen et al. (1976), p. 410.

Likewise as it occurred with the central focus of Dr. Livingston's investigation, it also took place with her complementary, collateral and most important research as well, the ABA capacity of neutralizing hCG. The central topic of Dr. Livingston's research was in certain form verified aftermath, but not her complementary or collateral research, which it is motive and impulse of this invention.

In 1984 Dr. Livingston published her book “The Conquest of Cancer”, in which she mentioned results on pp. 15-38, of applying in humans an integral treatment stimulating the immune system with several vaccines and administering ABA by a dietetic via. Among her vaccines, she used the one based in the Calmette-Guerin bacillus (Tuberculosis). No human being has been injected with ABA against cancer ever, just animals in vivo and human cells in vitro have received treatment.

Since Dr. Livingston's works, which were crystallized in researches, books and inventions, nobody else had studied and mentioned ABA in relation to cancer until 2005 and following years. During the year 2006, the invention number (CN 1748674A) published by The Chengdui Biological Institute Academy of Sciences, titled “New Use of Natural Abscisic Acid (ABA) in Developing Differentiation Inducer Drugs of Tumor Cells”, experimentally determined that ABA is able to: make proliferating tumor cells stagnate in S-phase and stop cell division, become cancer cells in normal cells, produce apoptosis and inhibit angiogenesis in a variety of tumor cells. In addition, on 2006 and re-examining the Livingston-Wheeler contentions, Marianne Ehrhorn Kruse, then in the Department of Biochemistry and Molecular Biology of the University of Southern Denmark, elaborated a master thesis titled “The Importance of Abscisic Acid as Possible New Drug in Cancer Treatment and its Role on Human Chorionic Gonadotropin Pathways”. She found that, ABA caused a tumor growth reduction, reduced cell proliferation rate, changed cell cycle progression, and produced induction of apoptosis, in four human cancer cell lines (HELA, DU145, HCT116 AND K562). In addition, ABA has also been proven in hepatocarcinoma cells and oral cancer.

ABA identification as an endogenous cytokine in human granulocytes and demonstration of ABA presence in human lymphocytes, fibroblasts, mesenchymal stem cells, platelets and monocytes has significant importance in its role for fighting cancer cells.

ABA as plant hormone could have been transferred to animals during the evolution, it could confirm: ABA serious implications in animal and human metabolism, and its identification as a new mammalian hormone. Previous knowledge of ABA plant induced effect has importance to explore such implications. Mostly, ABA has been studied around the world in connection to agricultural concerns, specifically about drought and water stresses. Since several years ago, ABA had been studied in relation to the phenomenon of the Global Warming, by several laboratories around the world, because it is necessary genetically to improve and develop new varieties of cultivates that, they can be able to produce adaptation to the changing temperatures and increased drought effect.

### OBJECTIVES OF THE INVENTION

1. Stimulate and encourage researchers to keep investigations going, about ABA and its properties against cancer.

2. Define probable mechanisms of ABA to fight cancer.

3. Produce technical information that, can be useful to make a clinical research and for manufacturing an adequate pharmaceutical medicine.

4. Verify and precise testimony expressed by Dr. Livingston, about ABA capacity to neutralize the hormone of cancer.

### DRAWINGS

Following drawings are presented in this invention:

FIG. 1 Molecular structure of ABA.

FIG. 2 Molecular structure of Sialic acids (SIA).

FIG. 3 Curve of mechanism of Na⁺ in proliferating normal cells.

FIG. 4 Concentration gradient of Na⁺ toward the extracellular fluid.

FIG. 5 Concentration gradient of Na⁺ toward the cell.

FIG. 6 Electroscope in contact with a negatively charged body.

FIG. 7 Electroscope in contact with a positively charged body.

FIG. 8 Curve of ABA uptake,

FIG. 9 Curve of a buffer solution.

FIG. 10 ABA absolute configuration and number of carbon atoms.

FIG. 11 phaseic acid.

FIG. 12 dihydrophaseic acid.

FIG. 13 lactone.

FIG. 14 1′,4′-cis-diol of (+)-abscisic acid.

FIG. 15 1′,4′-trans-diol of (+)-abscisic acid.

FIG. 16 (+)-xantoxin (2-cis).

FIG. 17 (+)-xanthoxin (2-trans).

FIG. 18 (+)-xanthoxin acid (2-cis).

FIG. 19 xanthoxin acid methyl ester (2-cis-isomer).

FIG. 20 (+/−) abscisic alcohol.

FIG. 21 (+/−)-abscisic aldehyde.

FIG. 22 ABA derivative XIII.

FIG. 23 ABA derivative XIV.

FIG. 24 ABA derivative XV.

FIG. 25 ABA derivative XVI.

FIG. 26 ABA derivative XVII.

FIG. 27 ABA derivative XVIII.

FIG. 28 ABA derivative XIX.

FIG. 29 1′-hydroxy-alfa-ionylidene acetic acid.

FIG. 30 ABA derivative XXI.

FIG. 31 ABA derivative XXII.

FIG. 32 ABA derivative XXIII.

FIG. 33 ABA derivative XXIV.

FIG. 34 ABA derivative XXV.

FIG. 35 ABA derivative XXVI.

FIG. 36 alfa-ionylidene acetic acid.

FIG. 37 ABA derivative XXVIII.

FIG. 38 alfa-ionylidene ethanol.

FIG. 39 ABA derivative XXXI.

FIG. 40 ABA derivative XXXII.

FIG. 41 ABA derivative XXXIII.

FIG. 42 ABA derivative XXXIV.

FIG. 43 ABA derivative XXXV.

FIG. 44 ABA derivative XXXVI.

FIG. 45 ABA derivative XXXVII.

FIG. 46 ABA derivative XXXVIII.

FIG. 47 ABA derivative XXXIX.

FIG. 48 ABA derivative XL.

FIG. 49 1′-deoxy-ABA.

FIG. 50 ABA derivative XLII.

FIG. 51 ABA derivative XLIII.

FIG. 52 1′-desoxy ABA methyl ester (ABA derivative XLIV).

FIG. 53 ABA derivative XLV.

FIG. 54 ABA derivative XLVI.

FIG. 55 ABA derivative XLVII.

FIG. 56 ABA derivative XLVIII.

FIG. 57 ABA derivative XLV.

FIG. 58 ABA derivative XLVII.

FIG. 59 ABA derivative XLVIII.

FIG. 60 ABA derivative XLIX.

FIG. 61 ABA derivative L.

FIG. 62 ABA derivative LI.

FIG. 63 ABA derivative LIIi.

FIG. 64 ABA derivative LIII.

FIG. 65 ABA derivative LIV.

FIG. 66 ABA derivative LV.

FIG. 67 ABA derivative LVI.

FIG. 68 ABA derivative LVII.

FIG. 69 ABA derivative LVIII.

FIG. 70 ABA derivative LIX.

FIG. 71 ABA derivative LX.

FIG. 72 ABA derivative LXI.

FIG. 73 ABA derivative LXII.

FIG. 74 ABA derivative LXIII.

FIG. 75 ABA derivative LXIV.

FIG. 76 ABA derivative.

FIG. 77. ABA beta-d-glucose ester.

FIG. 78 epimer-dihydrophaseic acid.

FIG. 79 2-trans ABA.

FIG. 80 ABA liophilic form.

FIG. 81 (+) 7′ hydroxy ABA.

FIG. 82 metabolite C (6′-hydroxy methyl ABA).

FIG. 83 (+/−) cis-trans ABA.

FIG. 84 (−) racemic ABA.

FIG. 85 (+/−) trans-trans ABA methyl ester.

FIG. 86 (+) trans-trans ABA.

FIG. 87 (+/−) cis-trans ABA methyl ester.

FIG. 88 (+) cis-trans ABA methyl ester.

FIG. 89 4-alfa-ionylidene acetic acid.

FIG. 90 gamma-ionylidene ethanol.

FIG. 91 4′-hydroxy-gamma-ionylidene acetic acid.

FIG. 92. 1′-4′-hydroxy-gamma-ionylidene acetic acid.

### REFERENCE NUMBERS

10 Electrochemical potential difference (EPD) with values between 0 and −70 mv.
12 Sodium concentrations in percent.
14 Isoelectric point.
16 Curve of intracellular sodium concentration [Na⁺]i.
18 Curve of extracellular sodium concentration [Na⁺]e.
20 Quiescent stage.
22 Mitogenesis.
24 Hyperpolarization.
26 Depolarization.
28 First layer (stern layer).
30 Cancer cells plasma membrane showing negative charges.
32 Cations attached to plasma membrane.
34 Second layer (diffuse layer).
36 Positive and negative ions in diffuse layer.
38 Third layer (hCG layer).
40 Negative charges of SIA.
42 Repulsed or attracted cations in third layer.
44 Metal ball of the electroscope.
46 Metal rod of the electroscope.
48 Metal leaves of the electroscope.
50 Isolating material or gasket.
52 Glass container of the electroscope.
54 Negative or positively charged body or ion.
56 Electrons.
58 Electron transfer.
60 Curve of ABA uptake.
62 ABA uptake concentrations in percents.
64 Values of pH between 3 and 8 in a medium outside plasma membrane.
66 Area of ABA uptake maximum efficiency for inducing stomata closure.
68 Area of ABA uptake maximum efficiency for inducing stomata opening.
70 pK of ABA (4.7).
72 Values of pH between 4 and 7.
74 Relative concentrations of carbonic acid (H₂CO₃) ranging between 0 and 100%.
76 Relative concentrations of HCO₃⁻ (bicarbonate) ranging between 0 and 100%.
78 pK of carbonic acid (6.1).
80 Curve of a buffer solution.
82 Region of maximum buffering capacity.
84 Normal blood pH.
86 ABA optimum extracellular range of pH for inducing cell normalization.
88 ABA optimum extracellular range of pH for inducing apoptosis.
89 Methyl

### DETAILED DESCRIPTION OF THE INVENTION

### ABA Natural Configuration and its Derivatives

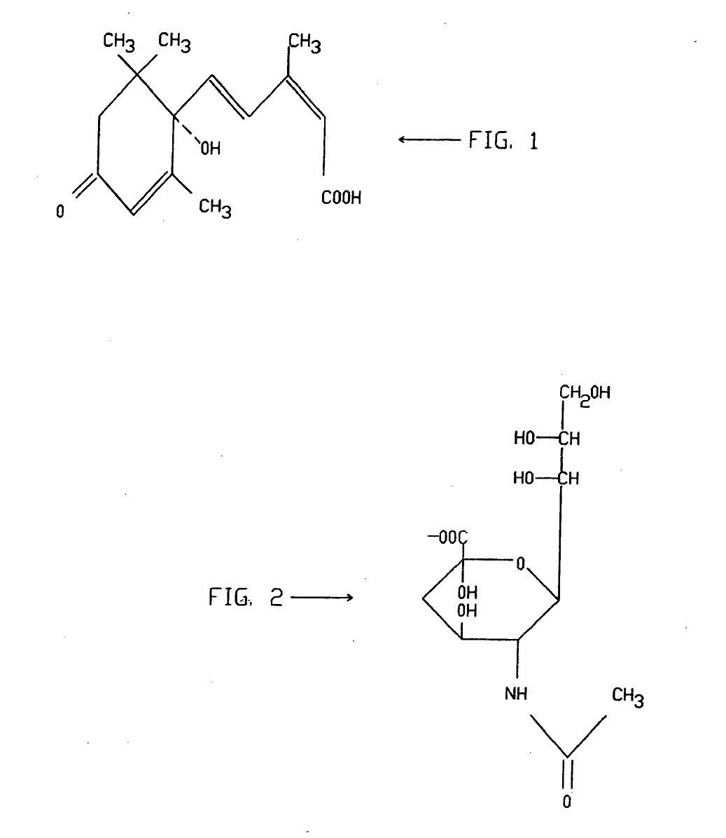

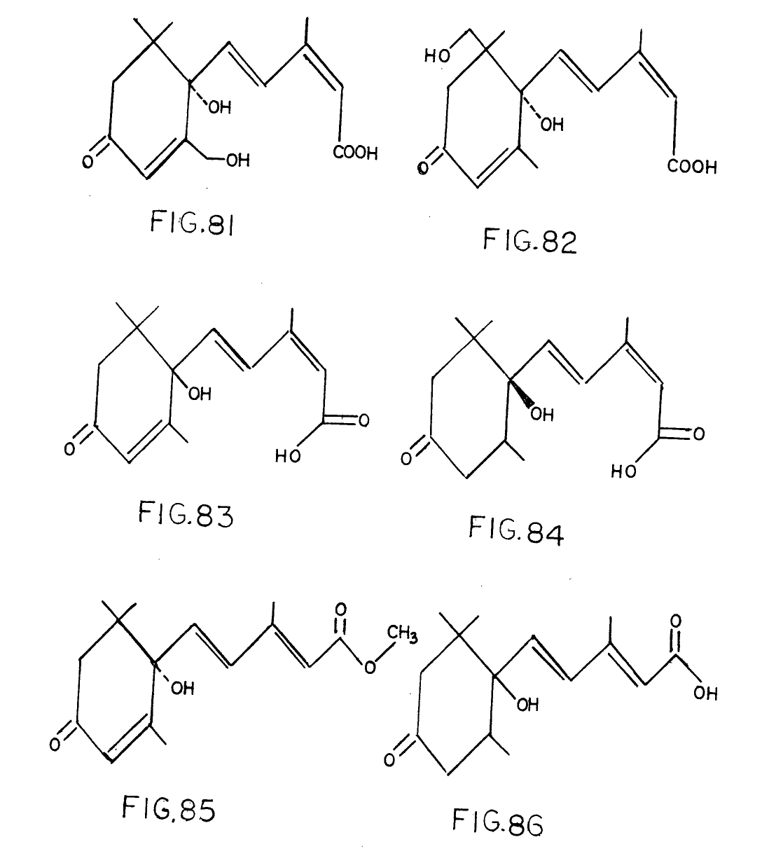

The ABA configuration and the location of the carbon atoms may be seen in the FIG. 10. ABA is a weak carboxylic acid and the carboxyl group is located in the carbon 1. ABA is also conformed by a cyclohexene ring (linking carbons atoms 1′ to 6′). The molecule also shows a hydroxyl group linked to carbon 1′ and oxygen (ketone) linked to carbon 4′. Four methyl groups of the molecule are located in carbons 3, 7′ and 6′. Natural ABA and its derivative unnatural form (racemic), which they can be seen in FIG. 1 and FIG. 84 respectively, differ just due to the extremely intense optical rotatory dispersion. The two sides of a plane through the optical center differ only in that, carbon-6′ carries two methyl groups whereas carbon-2′ carries one and one double bond. Consequently, it is possible for the hydroxyl group of the racemic (−) to occupy the same position as that of the natural form (+), if the cyclohexene ring lies on its other face and carbon-6′ and carbon-2′ are interchangeable. ABA natural and racemic form has a similar potency in relation to the biological activity, but natural ABA has an increased effectiveness in stomata response and in inhibition of root growth. FIG. 83 shows the ABA synthetic derivative (+/−), meantime the FIG. 84 shows the ABA enantiomer when racemic (−). FIG. 86 shows the ABA trans-trans natural material, where carboxyl group is positioned in trans configuration.

The rest of the ABA derivatives shows a reduced inhibitory effect. Any change in the ABA molecule decreases the growth-inhibitory activity (Milborrow B. V 1974). Such evidence demonstrates that, chemical structures shown herein are authentic ABA derivatives. ABA similar molecular structures of the derivatives also can be directly observed in figures of the drawings.

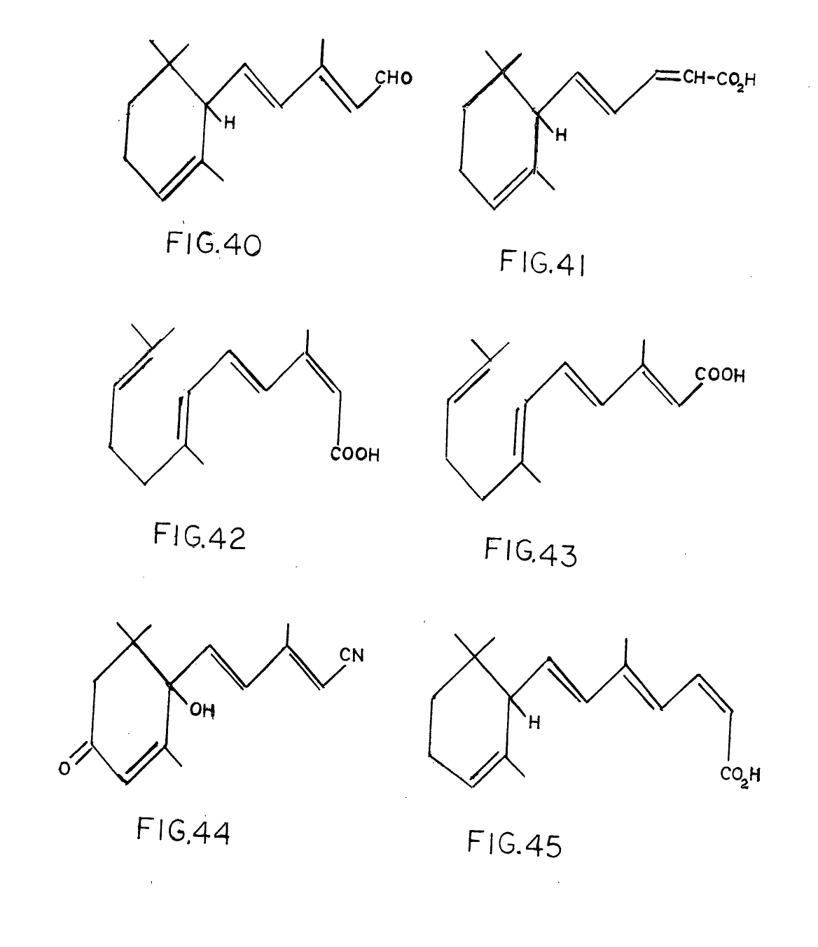

The inactive natural form 2-trans isomer of ABA (FIG. 51) and inactive synthetic form (+/−) (FIG. 79) are produced by isomerization of the 2-cis bond of ABA to give 2-trans ABA. Inactive ABA differs from the active, because the carboxyl group in the inactive form is moved to a north upper position and the hydrogen to a south lower position.

The mayor pathway by which ABA is metabolized, far involves the hydroxylation of one of the 6′ gem dimethyl groups of ABA (FIG. 76), followed by rearrangement to phaseic acid (PA) (FIG. 11), and reduction to dihydrophaseic acid-DPA (FIG. 12). In PA, an oxygen bonds carbon 6′ to carbon 2′. In DPA, in addition, the ketone is reduced forming a hydroxyl group and hydrogen. The epimer of DPA also could be formed, by reversing the positions of the hydroxyl group and hydrogen as seen in DPA (FIG. 78).

The reduction of the ketone in the ABA molecule gives two isomeric reduction products: 1′,4′-cis-diol of ABA (FIG. 14) and 1′,4′-trans-diol (FIG. 15).

The lactone molecule (FIG. 13) hydrolyzes to a 4-keto acid. It collapses on acidification, and the hydrogen on carbon 2 thereby, comes to be adjacent to a carbonyl group.

In FIGS. 16, 17, 18 and 19, natural xanthoxins are formed. In these molecules the hydroxyl group in carbon 1′ is replaced by oxygen which bonds carbons 1′ and 2′. Also, the ketone is replaced by a hydroxyl group. An aldehyde group is formed on the 2-cis bond in FIG. 16 and the same group is formed on the 2-trans bond in FIG. 17. In molecule of the FIG. 18 a xanthoxin acid can be synthesized by forming a carboxyl group on the 2-cis bond. In the ABA derivative of the FIG. 19, a methyl is attached to carbon 1, forming it the xanthoxin acid molecule. Although xanthoxins are in fact precursors of ABA, they herein has been considered ABA derivatives, because the structural similarities to the ABA molecule.

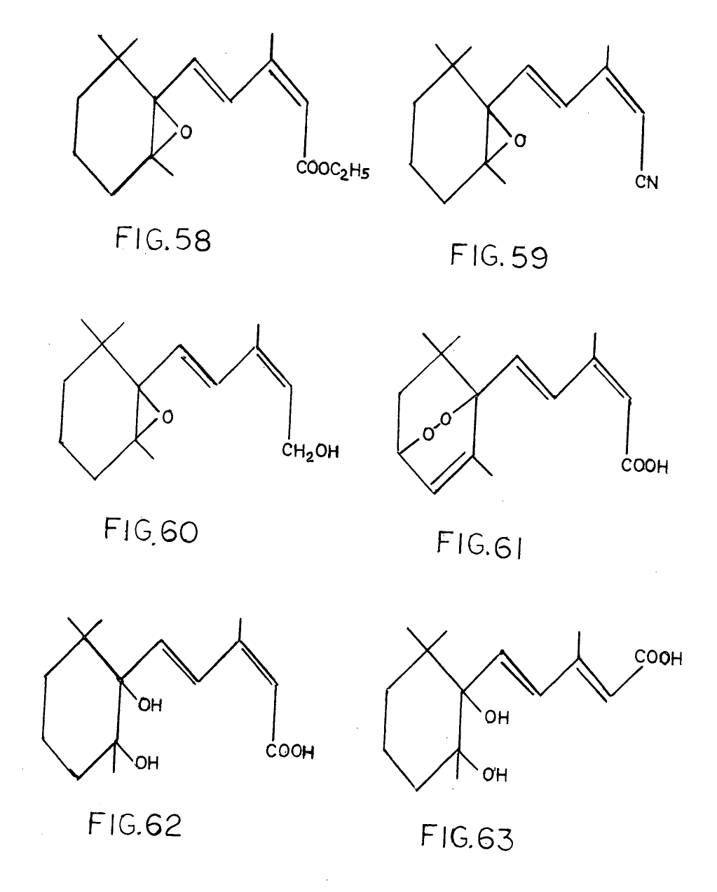

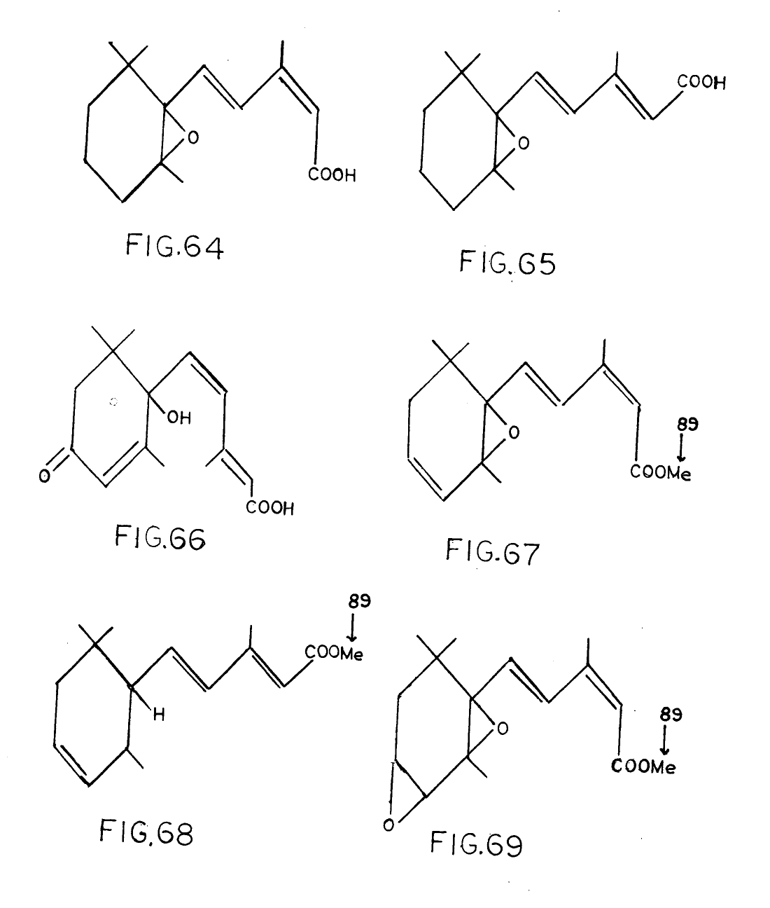

In FIGS. 64, 65 and 67 the hydroxyl group in carbon 1′ is oxidized and such change bonds an oxygen to carbons 1′ and 2′. In ABA derivatives of the FIGS. 64 and 65 the carboxyl group links, in both, to a 2 cis and a 2 trans position respectively. In ABA derivative of the FIG. 67, carboxylate group bonds to a methyl group. Those ABA derivatives are less oxygenated and less polar than ABA, and they have high capacity of penetration into the cells.

In ABA derivatives of the FIGS. 20 and 60, abscisic alcohol is formed by reacting the carboxyl group with an alcohol, in this case producing (methanol)-CH₂OH. In FIG. 60 an oxygen bonds carbon 1′ and carbon 2′. FIG. 48 shows the trans isomer of the abscisic alcohol of FIG. 20. In the ABA alcohol molecule of FIG. 90, the ketone in carbon 4′, the hydroxyl in carbon 1′ and the methyl in carbon 7′ are displaced producing a significant difference with the molecule of ABA in FIG. 1.

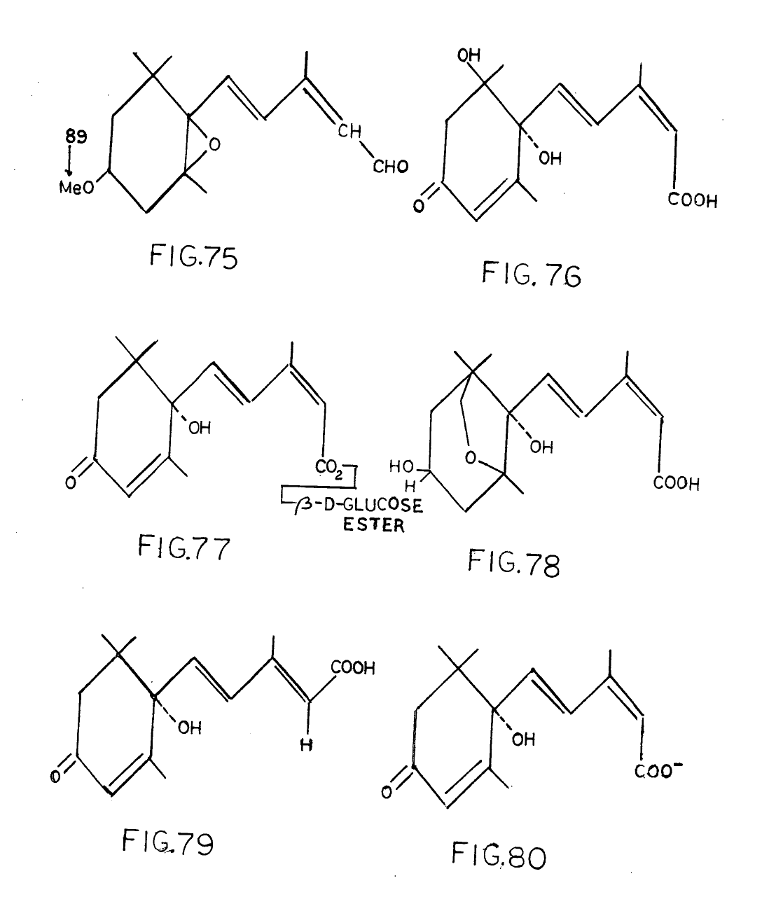

In the molecule of the FIG. 21, an abscisic aldehyde is structured in carbon 1 by forming an aldehyde group (—CHO). A similar structure of an abscisic aldehyde can be found in FIG. 39. In said structure already mentioned, the hydroxyl group is reduced in carbon 1′ and the ketone is reduced in carbon 4′. The trans isomer of FIG. 39 could be seen in FIG. 40. ABA derivative of FIG. 46 also shows an abscisic aldehyde. In this molecule, the spatial orientation of the aldehyde group bonds by following the same direction, as the hydrogenated carbon 2 to which is bonded. Abscisic aldehyde of FIG. 75 differs from that of FIG. 46, because in it, a hydroxyl is oxidized and a methyl is bonded to the ketone in carbon 4′.

The ABA essential molecule has two forms: polar (lipophobic), which can be seen in FIG. 80 and nonpolar (lipophilic), which can be seen in FIG. 1. Carboxyl group deprotonating or dissociation produces a carboxylate anion plus a hydrogen (FIG. 80). ABA lipophilic form can get across the plasma membrane, nevertheless, the lipophobic form (polar) can't.

The ABA Beta-D-glucose ester (FIG. 77) appears to be the form of rapid storage of ABA inside of cell, and also the ABA form of plant vascular transportation. In cytoplasm, an active form of ABA is released from ABA-glucose ester in plants by enzymatic hydrolytic reaction produced by beta-glucosidase.

Metabolite C (FIG. 82) was deduced to be the 6′-hydroxymethyl derivative of ABA. This compound is extremely unstable and can produce a rearrangement to PA (Milborrow B. V 1974).

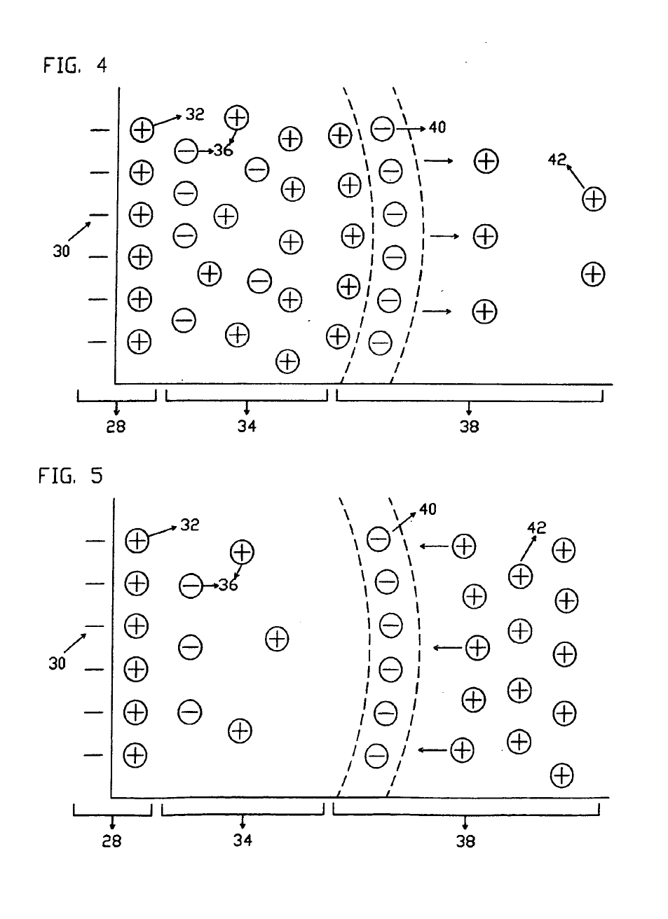

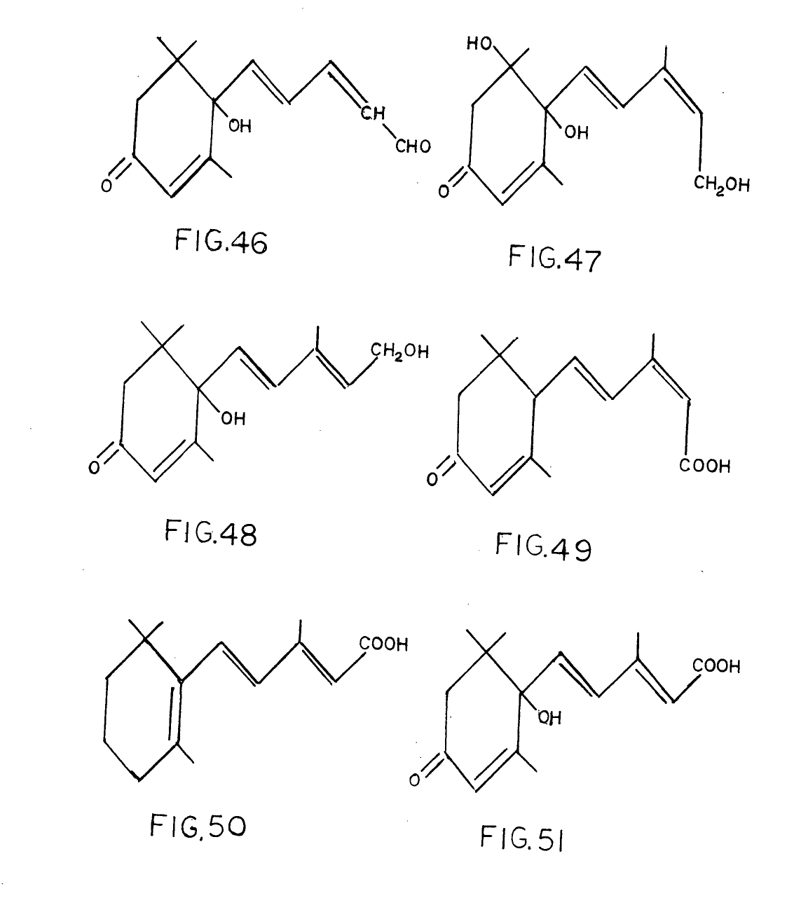

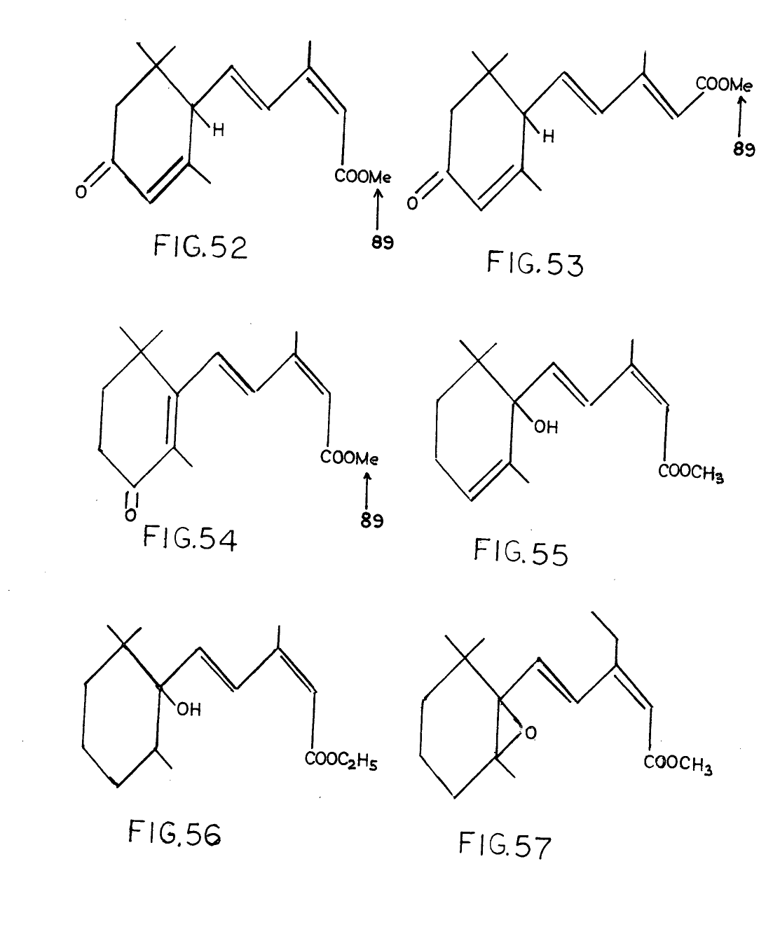

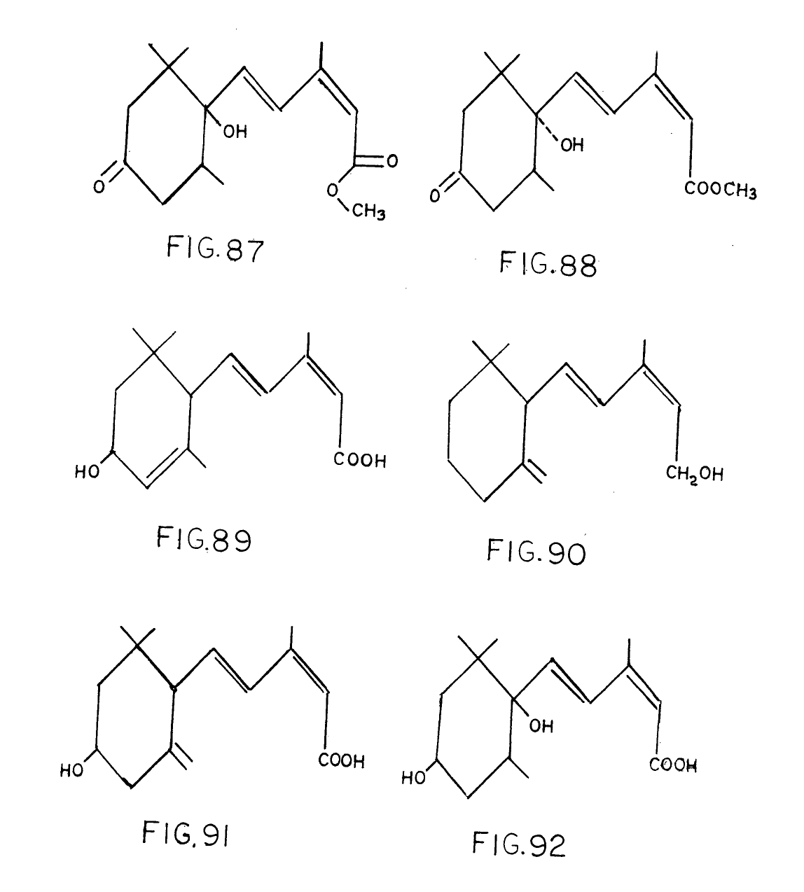

A diversity of ABA-methyl esters can be observed in FIGS. 52, 53, 4, 55, 57, 68, 69, 71, 85, 87 and 88. ABA methyl esters are originally generated by cleavage of the hydroxyl oxygen-hydrogen bond by reacting with an alcohol to form an ester. In FIG. 52 it is shown an ABA-methyl ester in cis configuration and its isomer in trans configuration (FIG. 53), both containing, a hydrogen located in carbon 1′. In FIG. 54 an ABA methyl ester in cis configuration shows the ketone group in carbon 3′. In FIG. 55 the ABA methyl ester is positioned in cis configuration and ketone is reduced. In FIGS. 57 and 67 the ABA-methyl ester in cis configuration contains oxygen linked to carbons 1′ and 2′. In FIG. 67 double bond of cyclohexene ring is located in carbon 3′. In FIG. 68 the ABA-methyl ester in trans configuration, contains a hydrogen in carbon 1′, and the ketone group of carbon 4′ is reduced. In FIG. 69 the ABA-methyl ester in cis configuration contains two oxygens linked to carbon 1′ and 2′, and in between 3′ and 4′. In FIG. 71 the ABA-methyl ester of FIG. 52 differs due to presence of a hydroxyl group, which is located in carbon 4′. In FIG. 85 a synthetic ABA methyl ester is almost identical to ABA of FIG. 1, but it differs due to ABA methyl ester group is located in trans configuration. In FIGS. 87 and 88, the ABA methyl ester group, natural and synthetic respectively, is located in cis configuration.

In FIG. 59, the presence of a cyano group may prevent the hydroxylation of the molecule or the other steps required to form ABA (Milborrow B. V 1974). In this structure, oxygen is linked to carbons 1′ and 2′. The cyano group (—CN) is located in cis configuration and the nitrogen in the group is linked to carbon 1. In ABA derivative of FIG. 44, a cyano group is also present. In this molecule the cyano group replaces the carboxyl group of ABA in carbon 1, but in trans configuration.

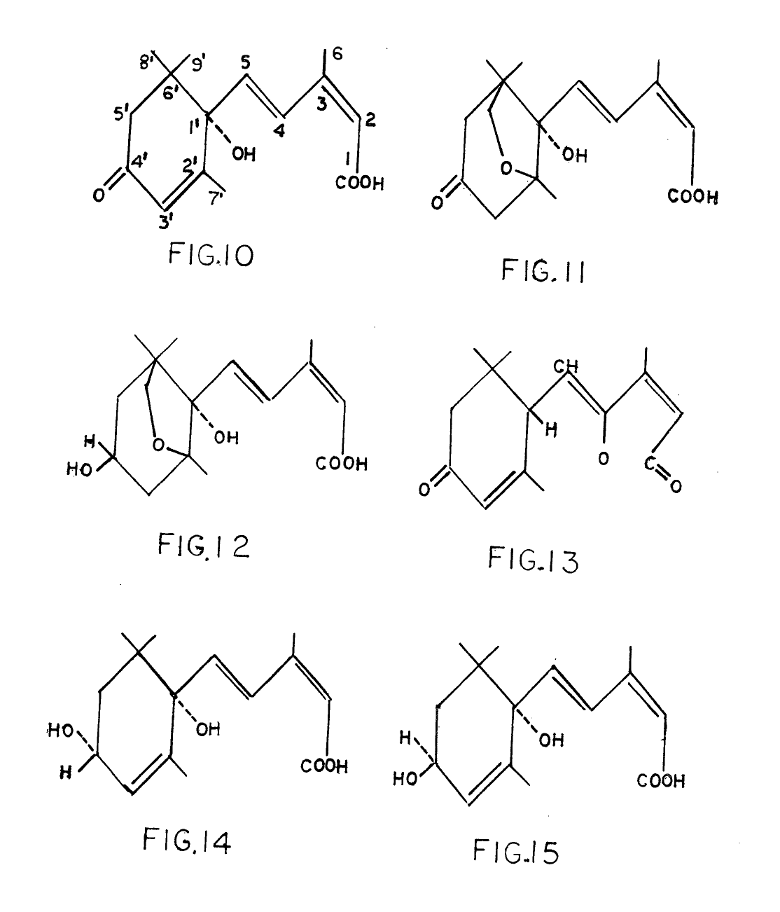

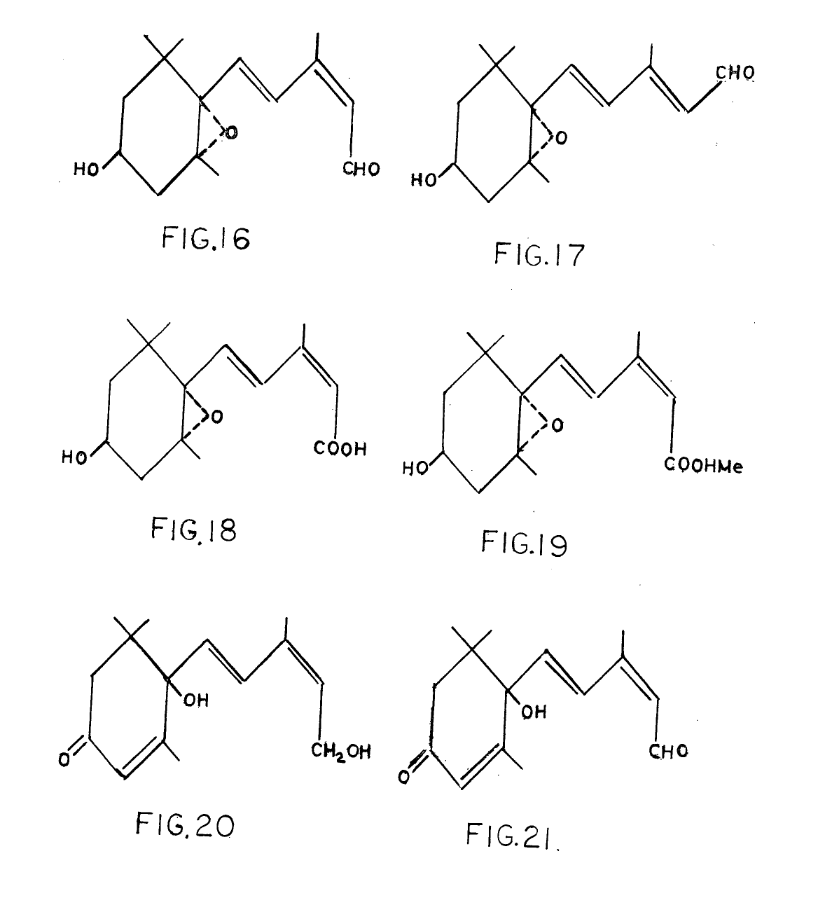

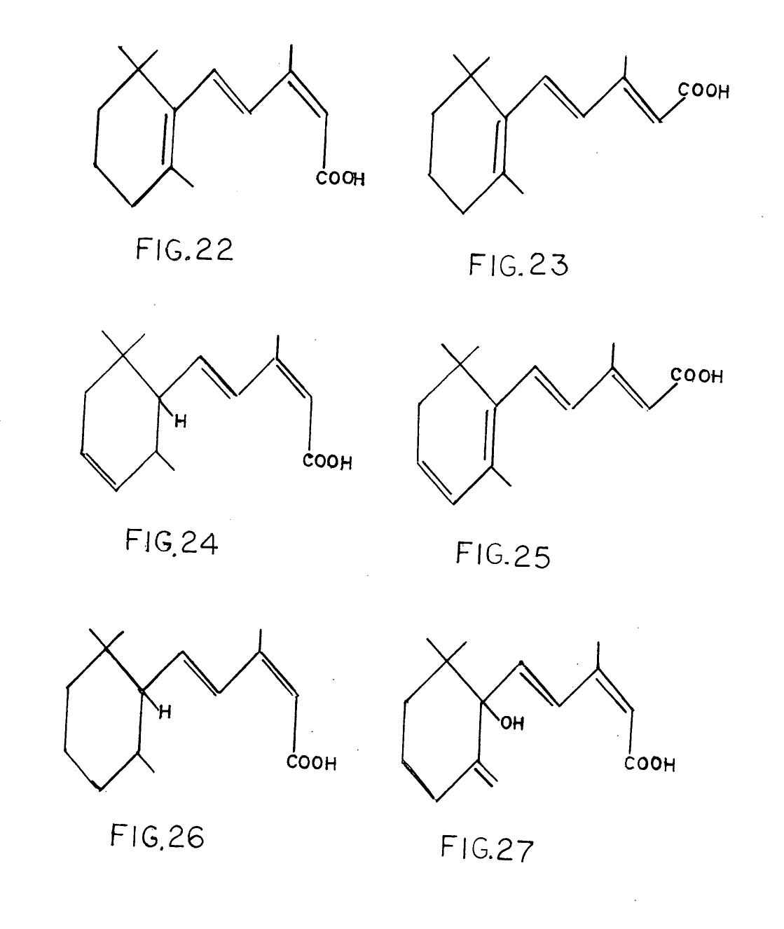

FIGS. 22 and 23 show molecular structures of ABA derivatives in which hydroxyl and ketone groups are reduced and double bond is present in carbon 1′, instead located in carbon 2′, as it can be seen in ABA molecule of FIG. 1. Both structures differ in the position of the carboxyl group. In ABA derivative XIII of FIG. 22, carbon 1 shows a cis configuration and ABA derivative XIV produces an isomer in trans configuration. ABA derivative of FIG. 25 differs from the one shown in FIG. 23, because the first one has a additional double bond in carbon 3′ of the cyclohexene ring.

FIGS. 24 and 26 show ABA derivatives with similar structure to ABA, but they do differ by containing a hydrogen in carbon 1′, instead an OH—. Likewise, ketone group in both molecules is reduced. In FIG. 24, double bond is present in carbon 3′. In FIG. 26 double bond is reduced in cyclohexene ring.

FIGS. 27 and 29 show a pair of derivatives with ABA similar structure as seen in FIG. 1, but they differ because ketone is reduced. In FIG. 27 the cyclohexene ring shows a double bond in carbon 2′. ABA derivative of FIG. 30 represents the trans isomer structure of the one seen in FIG. 29. According to Zeevaart J A D et al. 1988, such molecule of FIG. 30 is metabolized to ABA and t-ABA respectively. The ABA derivative of FIG. 29 is denominated 1′-hydroxy-alfa-ionylidene acetic acid and it is considered a minor product of the alfa-ionylidene acetic acid of FIG. 36.

ABA derivatives of FIGS. 28 and 31 are roughly similar to ABA of FIG. 1, but they are molecules shorten by two carbons atoms. In both structures, which they show a trans configuration, the carboxyl group is located in carbon 3. In structure of FIG. 28, ketone is reduced, meanwhile in FIG. 31, such group is present.

ABA derivative structure of FIG. 32 shows a double bond, linking carbon 3 to the carboxyl group. The cyclohexene ring is identical as shown in ABA molecule of FIG. 1. In ABA derivative of FIG. 33, it is located a triple bond which links carbon 4 and carbon 5, and carboxyl group is structured in trans configuration.

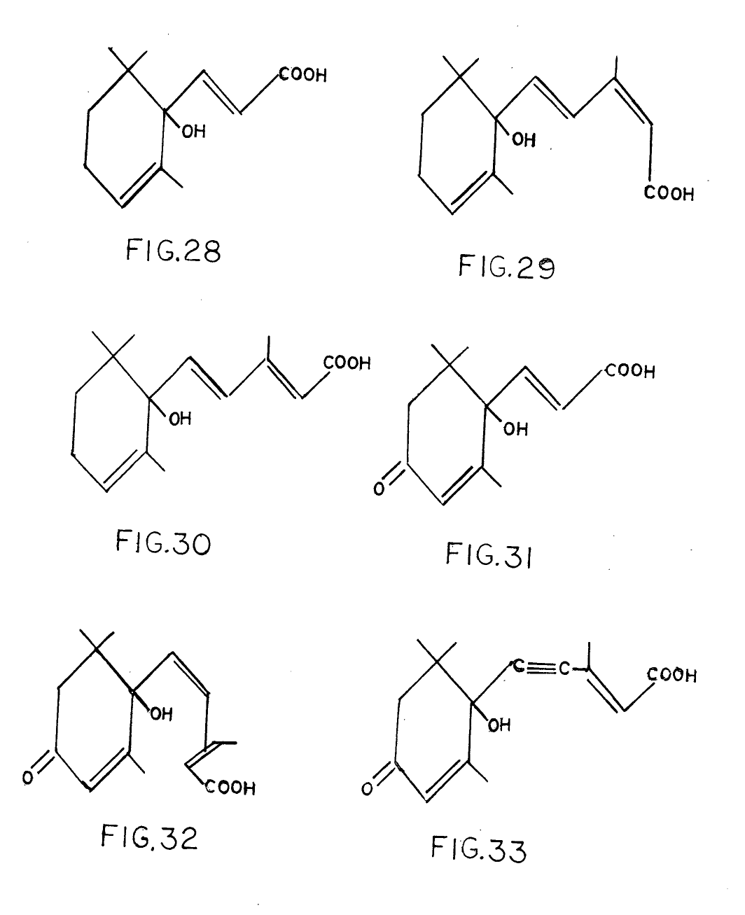

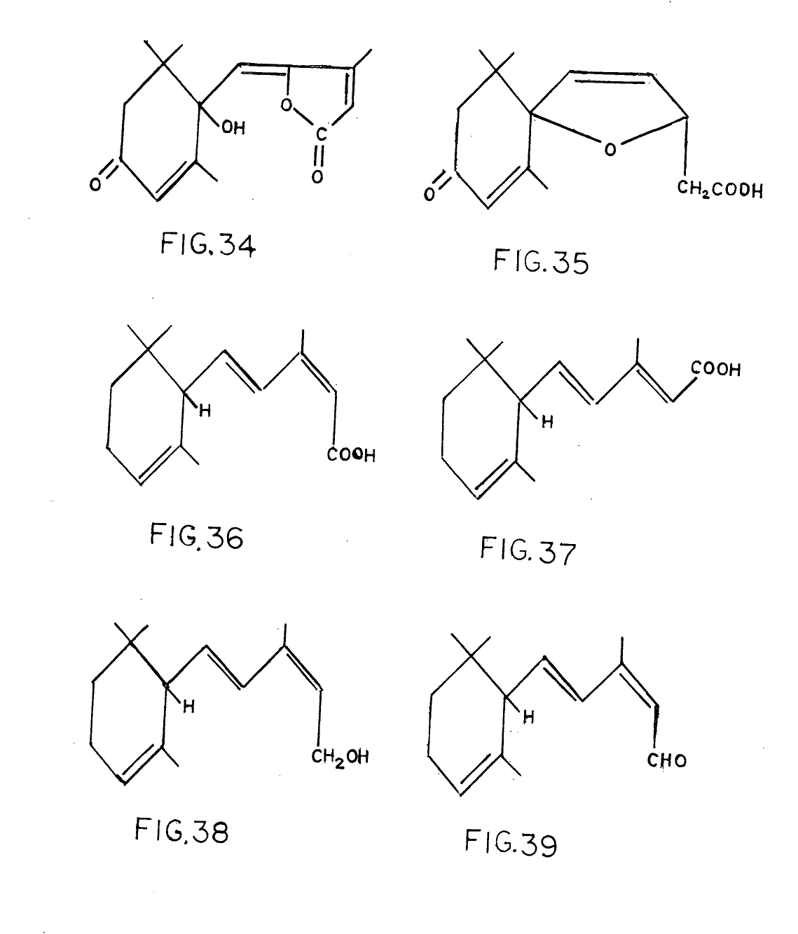

ABA derivative of FIG. 34 shows a penta ring, where carbon 1 is linked to an oxygen, and another oxygen bonds carbon 4 to carbon 1, producing cyclization. In this molecule, a hydroxyl is found in carbon 1′ and a ketone in carbon 4′. In molecule of FIG. 35 an etanoic acid (—CH₂COOH) is located in carbon 1, and an oxygen bonds carbon 2 to carbon 1′ forming a penta ring.

Evidence indicates that ABA could be formed from alfa-ionylidene derivatives, such as the ones shown in FIGS. 36 and 38 (Zeevaart J A D et al. 1988). The molecule of FIG. 36 is similar to ABA, but it differs because ketone is reduced in carbon 4′ and hydrogen replaces the hydroxyl group in carbon 1′. The trans isomer of the alfa-ionylidene acetic acid of FIG. 36 is shown in FIG. 37. Molecule of FIG. 38 is denominated alfa-ionylidene ethanol.

In FIG. 41, the ABA derivative shows the carboxyl group in trans configuration bonded to a hydrogenated carbon 2. In the molecule the ketone is reduced and hydrogen replaces the hydroxyl group. In FIG. 42 (cis configuration) and 43 (trans configuration), the ABA derivatives show a broken bond between carbon 1′ and carbon 6′. The structures also show double bonds in carbons 1′ and 5′.

In ABA derivative of FIG. 45, the side chain has two more carbons than the ABA molecule of FIG. 1. This structure replaces the hydroxyl group usually found in the ABA molecule by hydrogen and ketone is reduced.

According to Zeevaart J A D et al. 1988, when mevalonic acid is fed to the fungis Cercospora rosicola the main products are: ABA and the molecule of FIG. 49 (1′-deoxy-ABA). In this ABA derivative, hydroxyl group of carbon 1′ is reduced.

In FIG. 50, the carboxyl group is conformed in trans configuration, the hydroxyl and ketone are reduced, and double bond in cyclohexene ring is present in carbon 1′.

FIGS. 56 and 58 show ABA derivatives of ethyl esters (—COOC₂H₅). Specifically in FIG. 56 the molecule preserves the hydroxyl group in carbon 1′; meantime in FIG. 58 said hydroxyl group is oxidized. FIGS. 72, 73 and 74 show different variations of ABA ethyl esters. In FIG. 72, the ABA ethyl ester includes a methyl bonded to the ketone in carbon 4′. The ABA derivative of FIG. 73 differs of molecule of FIG. 72, because an oxidation replaces the hydroxyl group in carbon 1′ in FIG. 73. ABA derivative of FIG. 74 is reduced in carbon 1′ by hydrogen, and in carbon 4′ by a hydroxyl.

In ABA derivative of FIG. 61, the molecule is oxidized. Two oxygen bond carbon 1′ and carbon 4′ in the cyclohexene ring. In FIGS. 62 and 63 a hydroxylation replaces the methyl group in carbon 2′ and a double bond in carbon 2′ is replaced by a hydrogenated bond. FIG. 63 shows the trans steroisomer of molecule in FIG. 62.

In both ABA derivatives of FIGS. 64 and 65, the molecule is oxidized. The hydroxyl group is replaced by oxygen, which bonds carbon 1′ and carbon 2′ in cis and trans configuration respectively.

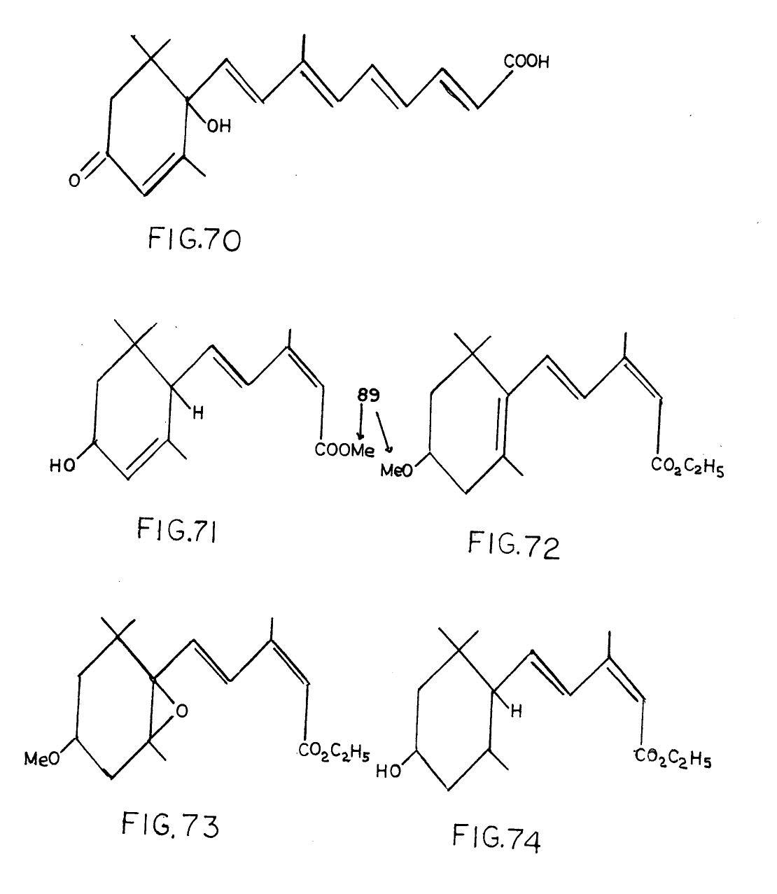

In FIG. 66 the ABA molecule produces a isomer where the carbons 2 and 3 of the side chain are oriented south, preserving the carboxyl group, which is bonded to a double bond. In ABA molecule of FIG. 70 the side chain contains 9 carbons, four more than the side chain of the ABA molecule of FIG. 1. In this ABA derivative, the cyclohexene ring is preserved intact as ABA in FIG. 1.

In (+) 7′ hydroxy ABA of FIG. 81, the molecule is oxidized when a hydroxyl group replaces the methyl group in carbon 7′. This molecule has been found by researchers as a minor and transient product after some species of plants are fed with natural ABA or racemic.

In ABA derivatives of FIGS. 89, 91 and 92, the molecule is reduced and a ketone group is replaced by a hydroxyl. In molecules of FIGS. 89 and 90 the hydroxyl of carbon 1′ is also reduced, but in ABA molecule of FIG. 92, said hydroxyl is preserved in carbon 1′.

### ABA in Relation to the Human Chorionic Gonadotropin

Dr. Livingston postulated in her U.S. Pat. No. 3,958,025 (1976), col 8, line 50, that a hormone immunologically identical to hCG denominated Microbic Chorionic Gonadotropin, expressed from Progenitor cryptocides, might be opposed or neutralized by a growth retardant in vitro. Such a growth inhibitor was identified as ABA.

Experiments in vivo of Dr. Livingston, mentioned in her patent (col 9), demonstrated capacity of ABA to neutralize hCG. For determination of cancer survival rate, she used C57BL/6J mice and C1498 transplanted tumor with myeloid leukemia purchased from the Jackson Laboratory in Maine. This type of cancer was lethal in mice in 10-15 days. ABA furnished by Hoffmann-Laroche was suspended in saline and administered as suspension. Groups of 10 mice were used for treatment for a total of 7 days.

The following rate of survival was noted at the end of 14 days: Group I: control saline intraperitoneal (i.p)=3 survivors; Group II ABA 1 mg/kg (i.p)=9 survivors; Group III ABA 10 mg/kg (i.p)=10 survivors; Group IV ABA 10 mg/kg (oral)=6 survivors; Group V ABA 100 mg/kg (oral)=9 survivors.

It was concluded that ABA has a marked effect in the inhibition of mice (C57BL/6J) with the tumor system (C1498).

In 1984, Dr. Livingston obtained, pp. 15-38, after treatment of 62 random cases in humans with cancer a success rate around 82%, not considering it inconclusive cases. She applied only a digestive treatment with ABA plus an elimination of the bacterium P. cryptocides with her set of six vaccines.

Since 70 years ago, Agrobacterium tumesfaciens was identified as the cause of crown gall disease which is characterized by formation of neoplasm (galls) in plants. Beijersbergen et al. 1992, p. 1324, determined that such bacterium causes the disease by a DNA transfer. Some others bacteria have been found in plants causing the cancer disease: Pseudomonas syringae subsp. savastanoi (in olive and oleander) and Erwinia herbicola pv. gypsophilae (in Gypsophila), cited in Ullrich C. I et al. 2000.

Actually it is recognized that, bacteria are not the only agents for inducing cancer; viruses, chemical compounds (toxins), physical elements such as prolonged exposition to solar radiation and other factors can provoke the disease.

Appearance of hCG in tumor cells, induced or not by a specific agent as bacterium or it occurring in placenta or membranes of sperm cells, is a natural mechanism for protecting foreign cells against the immune system of a host organism. hCG is a sialoglycoprotein hormone produced by the human placenta, having by function maintenance of the steroid hormone secretions of the corpus luteum and protecting the embryo and fetus against the immune system of the mother. A medicine against cancer must have a fundamental property of counteracting hCG to facilitate viability of tumor destruction by the immune system.

hCG is a glycoprotein containing oligosaccharides. The hormone is composed of two subunits, alfa and beta subunits, having a total of 244 amino acids and forming a complex associated molecule with the cellular membrane.

According to Acevedo 2002, pp. 135-136, alfa subunit contains two chains of n-linked oligosaccharides attached to asparagine with two molecules of n-acetyl-neuraminic acid, known as SIA (FIG. 2). The beta subunit contains four chains of o-linked oligosaccharides attached to the four serines of the hCG beta carboxy-terminal peptide with a total of six molecules of SIA.

The high content of SIA gives the membrane-associated hCG molecule a very high negative charge. SIA appears to be the regular components of all types of mucoproteins, mucopolysaccharides and certain mucolipids.

Cells from the human immune system express in their membranes, normal negative charges. Equal polarity of hCG and immune system cells make such cells immunologically inert and unable to get close and attack tumor cells (Acevedo 2002, p. 136). Specific inhibition of human natural killer cell by SIA and sialo-oligosaccharides has been: researched by Van Rinsum et al. 1986, and published through the International Journal of Cancer, vol 38, pp. 915-922.

In addition to the mentioned cancer blockade created by repelling charges against immune cells, hCG also stimulates malignant growth which is summarized in the abstract 227 of Raikow et al. 1987, p. 57, issued in the Annual Meeting of the American Association for Cancer Research.

Others researchers as Stern et al. 1999, p. 367, have related increased negativity in cancer cell plasma membrane with a loss of electrons and protons toward extracellular space. This loss of electron/proton homeostasis and reversion to a glycolytic state are esteemed the basis of their proposed model of carcinogenesis. In this model, DNA abnormalities are considered “contributory or secondary phenomena”.

### ABA in Relation to Cancer Metabolism and Ion Transport

According to Van Slyke 1933, p. 184, from one third to three-fourths of the mineral base in the cellular cytoplasm in normal cells must be neutralized by complex acids, chiefly the proteins and the phosphatides, which are alike in being buffers and in diffusible colloids. Bases such as K⁺ and Na⁺ are anchored in the cells in which they form components. Such confrontation of charges, negative and positive, is denominated redox centers (see ahead).

According to A. Keith Brewer 1984, p. 1, in his research named, “The High pH Therapy for Cancer, Test on Mice and Humans”, mass spectrographic and isotope studies have shown that K⁺, Rb⁺ and specially Cs⁺ are most efficiently taken up by cancer cells. Elements such as K⁺ exert important functions in cancer cell, as for example, it transports glucose into the cell (Brewer 1984, p. 2). K⁺ is also important in actively proliferating growing tissues such as embryonic and cancer cells (Delong et al. 1950, p. 721).

Despite above mentioned statements about K⁺, cancer cells have lower K⁺ concentrations and higher Na⁺ and water content than normal cells (C. Cone Jr, 1974). Likewise, Paul Seeger et al. 1990, cited in Haltiwanger 2003, p. 9, in the monograph, “The Electrical Properties of Cancer Cells”, sustains that, cancer cells have altered their membrane composition and permeability. This result in movement of K⁺, Mg²⁺, and Ca²⁺ out of the cell, and accumulation of Na⁺ and water inside. According to K. Brewer 1984, K⁺ transports 7 water molecules and Na⁺ transports 16 water molecules. Na⁺ accumulation in cancer cells brings an additional increase of water. It changes metabolism, physical size, and form of cell. Carcinogenesis causes that, original normal cell gets a round shape because an excess of water.

Apparently, malignant cell transformations have caused a differential preference of cations in relation to the negative charges concentrated in surfaces of the intracellular protein matrix.

G. N. Ling initiated this line of investigation, about the mentioned above differential preference of cations in cancer cells in the 1960's (Cope, 1978, p. 466), who called it the association-induction hypothesis. Cope called such phenomenon the tissue damage syndrome, because damage in any tissue, produces a similar set of changes in salt and water content. According to Ling, proteins of cells are able to exist in either of two different configurational states: a normal configuration, and a damaged configuration In a normal state, negatively charged sites on the protein matrix have a large preference for association with K⁺, rather than Na⁺, and cell water is highly structured. The result is high cell K⁺ and low cell Na⁺. In the damaged cell, proteins lose preference for association with K⁺ rather than Na⁺, also they lose their ability to structure water, with the result K⁺ leaves the cell, and is replaced by Na⁺, and the water content of the cell increases. Recent studies of cation-specific interactions with protein surfaces have indicated a strong preference of Na⁺ over K⁺ binding to surface of phosphates and carboxylate groups (Hess et al. 2009). It means that, Na⁺ must be actively pumped out of the cell to: keep an osmotic balance inside and outside of cell, preserve a cell aerobic metabolism and maintain a cell normal configuration state. Inhibition or activation of the Na⁺—K⁺ pump ATPase enzyme plays a significant role in a determined presence or preference of those cations in normal or cancer tissue.

On the other hand, Ca²⁺ in cancer cells is contained only at about 1% of that in a normal cell (K. Brewer 1984, p. 2). Likewise, Delong et al. 1950, p. 718 and Ambrose et al. 1956, p. 576, reveal that, tumor tissues show a decreased Ca²⁺ content in comparison with normal tissues. According to K. Brewer, Ca²⁺ transports oxygen into the normal cell. ABA increases Ca²⁺ in cytoplasm being taken from the extracellular medium. A Ca²⁺ and oxygen increase in cell, has a direct connection to the phenomenon of apoptosis. Limited absorption of Ca²⁺ is another cause of the anaerobic metabolism of cancer cells pointed out by Otto Warburg 1925.

Cancer anaerobic glycolytic metabolism, also denominated fermentation, which occurs during the first stage of cancer, is exclusively supported by the biochemical mechanism of glycolysis. It degrades glucose from blood producing lactic acid and alcohol. An anaerobic cell can only produce two ATP'S from the metabolism of a glucose molecule, while those aerobic cells can derive 36 ATP'S from burning a glucose molecule. That minimal energy provokes: a lack of cellular division and an increased necessity for glucose. It obligates cancer cells to consume bigger quantities of glucose to maintain a reduced state of metabolism. Such condition makes cancer tissues very addictive to glucose. Cancer cell metabolism changes during the progression of the disease, from an anaerobic in the beginning, as definited by O. Warburg, to an aerobic metabolism. The latter metabolic condition is a response caused by a significant oxygen supply coming the bloodstream and it is used by cancer cells to increase a rate of cell proliferation during the last stages of cancer.

During a malignant cell progression, oxygen pressure increases and vascularization develops. Nevertheless, cancer cells survive during aerobic glycolysis. This phenomenon is known as the Warburg effect. According to Newington J. T et al. 2011, in aerobic glycolysis pyruvate dehydrogenase kinase (PDK) is activated, instead of pyruvate dehydrogenase (PDH). In normal cells and in presence of oxygen, mitochondria converts pyruvate into acetyl-coenzyme A by PDH. In aerobic glycolysis, PDK represses mitochondrial respiration and forces the cell to rely heavily on glycolysis, even in the presence of oxygen. During a stage of aerobic glycolysis, a metabolic threshold could hypothetically define in what point of the cancer progression, the cell could be forced to reverse toward a normal condition. Twice nominated to the Nobel Prize P. Seegert demonstrated that, cancer cell can reverse toward a state of normal.

During the progression of the disease, cancer cells are able to switch its energy metabolic system by producing ATP through mitochondria and taking pyruvate and lactate to the Krebs cycle. It is denominated the “reverse Warburg effect”. Such phenomenon could be a significative cancer response to vascular development and oxygen efflux to tumors. Pavlides S et al. 2009, from the Thomas Jefferson University in Philadelphia, came out with the hypothesis that, epithelial cancer cells induce aerobic glycolysis in neighboring stromal fibroblasts. These cancer-associated fibroblasts undergo myoi-fibroblastic differentiation, and secrete lactate and pyruvate, which are taken up by cancer cells and used in the mitochondrial TCA cycle, thereby promoting efficient energy production. Still, cancer cells can go further, whether glucose is no longer available, as it can occur in solid tumors; cancer cells can be forced to use an alternative energy substrate, such as the oxidation of glutamine, a process called glutaminolyosis (Rossignol R. et al. 2004).

### ABA as Inhibitor of Alfa-Amylase

This hormone is also able in plants to inhibit enzymes as alfa-amylase by blocking hydrolysis of starch and interfering in supplies of glucose. Milborrow B. V 1967 concluded that ABA might be the major component of inhibitor-beta. Likewise Hemberg et al. 1961, pp. 861-867, showed that Abscisin II suppressed an activity of alfa-amylase, but only insignificantly affected an activity of beta-amylase in resting potato tubers. In addition, it has been suggested that, a starch disappearance in guard cells occur simultaneously with K⁺ entry (Manfield 1971, p. 147). During stomata opening ABA is inactivated, inhibiting the effect on alfa-amylase, but during stomata closure ABA inhibits alfa-amylase. This effect is produced in stomata movements, because glucose also acts as a solute osmotically active. According to Zeevaart Jan A. D. et al. 1988, page 462, when ABA is applied in barley aleurone layers to 25-fold excess of GA, the synthesis of alfa-amylase, protease, beta-glucanase, and ribonuclease is suppressed. So, alfa-amylase inhibition blocks an available glucose to the cell. Herein, it might be suggested that, ABA could induce a similar signal in animal cells, producing it a mechanism of glucose starvation. Further research, about this specific aspect must be developed.

### Na⁺ Mechanisms in Normal Cells

Researches of C. Cone Jr, former scientist of NASA (1974), clarified a relation between EPD, also called electrical transmembrane potential (Eₘ), and associated ionic concentration differences in mitogenesis control of normal and cancer cells.

It has been pointed out that, a cell multiplication or mitosis is stimulated by a cell depolarization. This phenomenon of potential fall is caused by Na⁺ concentration increase in the cytoplasm.

According to C. Cone Jr 1974, pp. 423-424, as density of cells increases, a substantial direct cell to cell surface contact begins to develop as well. Hence, a mitotic activity begins to decrease with a corresponding rise in membrane EPD.

Likewise, in order to get a quiescent stage, cells increase membrane EPD by decreasing Na⁺ concentration in cytoplasm.

In this invention, experiment of C. Cone Jr 1974, has been interpreted by applying a derivation of the Nernst equation, as follows:

EPD [mV]=61 log [Na⁺]e/[Na⁺]i.

Where, EPD is measured in mV; [Na⁺]e is an extracellular Na⁺ concentration and [Na⁺]i is an intracellular Na⁺ concentration.

In such experiments of Cone, Na⁺ and K⁺ were measured, therefore in agreement to his consideration, these concentrations could not be directly related to the measured Eₘ. Nevertheless, one ion concentration can be related to EPD, whether it is applied the already mentioned equation according to the concept of Nernst Potential. This concept, considers only one type of ion in a cell, where there would not be any other ions, to know distribution of such ion for a determined amount of potential.

EPD equation is derived from the Nernst Potential equation as it follows:

V=RT/z F ln(Co/Ci)

Where V=Nernst Potential in mV; R=universal gas constant (8.314 J/mol·K); T=absolute body temperature (321K); z=charge on ion; F=Faraday constant (96485.309 C/mol); In or Log of natural numbers=2.303 Log₁₀; Co=concentration outside membrane; and Ci=concentration inside membrane.

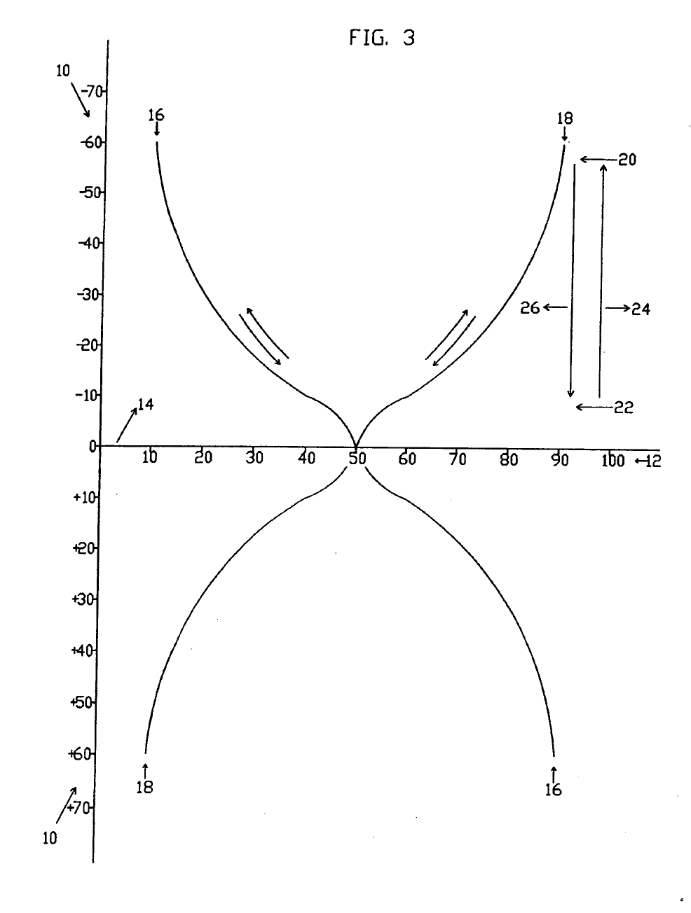

In FIG. 3, it is possible to observe a coordinate system, where y-axis is represented by the variable EPD (10) and x-axis is defined by Na⁺ concentrations in percent (12).

Two curves define a double function. One curve is related to [Na⁺]i (16). The another one represents to [Na⁺]e (18). In the upper plane, a depolarization is characterized by an increase in [Na⁺]i (16) and a decrease in [Na⁺]e (18). When EPD (10) gets lower values the cell is able to switch from the cell quiescent stage (20) to the cell mitogenesis stage (22).

In Cone's investigation 1974, pp. 423-425, it can be observed Na⁺ and K⁺ concentrations (mcmol/ml) in the mitogenesis stage (log phase) and the quiescent stage (saturated) for the used types of normal cells (CHO and 3T3). Values of EPD varied between −10 mV in mitogenesis stage and −65 mV in quiescent stage.

Experimental results by Cone 1974 are shown in the next table:

| | CHO MONOLAYER CELLS | | 3T3 MONOLAYER CELLS | |
|---|---|---|---|---|
| | MITO- | | MITO- | |
| ION | GENESIS | QUIESCENT | GENESIS | QUIESCENT |
| Na⁺ | 15.3 +/− 1.8 | 7.9 +/− 2.1 | 17.6 +/− 1.5 | 8.6 +/− 0.8 |
| K⁺ | 186.1 +/− 5.3 | 185.9 +/− 6.2 | 204.5 +/− 3.6 | 197.0 +/− 4.8 |

In these experiments, it may be seen that, a significant variation occurs in Na⁺, but not in K⁺. It means that, it is Na⁺ the ion really involved in cell mitogenesis control.

This experiment helps to understand why ABA is able to produce seed dormancy and growth inhibition in plants and also growth inhibition in animal cells. Therefore, it is evident and suggests that, ABA effect of cell hyperpolarization and decrease of [Na⁺]i in cancer cells, could be effects conducting reactive proliferating cells to get a quiescent or resting stage or normalization.

EPD and Na⁺ concentration changes, outside and inside of cell, are provoked and dependently related to, which Na⁺ transport system is activated and which one is inhibited. During the quiescent stage, normal cells depend of Na⁺—K⁺ pump ATPase enzyme. This enzyme mediates the transportation of 2K⁺ going into the cell for 3 Na⁺ going out of cell with the consumption of 1 ATP molecule in the process. Nevertheless, when a cell triggers mitosis a secondary transport system is activated. According to Mahnensmith et al. 1985, normal cells contain a secondary transport system that mediates the trans membrane exchange transport of Na⁺ for H⁺. It is also revealed in this research that, this secondary system plays among other, a physiological role in cell growth and proliferation.

### Na⁺ Mechanisms in Cancer Cells

C. Cone Jr 1974, p. 431, remarked that, “a cancer cell multiplication or mitosis is characterized by a sustained and pronounced cell depolarization, in conjunction to an increase of Na⁺ concentration in the cytoplasm. This phenomenon of malignant proliferation blocks or negates the effective functioning of the ionic regulatory system, resulting in a sustained cell depolarization with associated inability to lower the Na⁺ concentration to nonmitogenic levels. That cancer cell inability to decrease Na⁺ would be associated to a reduction of the effective operation of Na⁺ pump”. In cancer cells, Na⁺—K⁺ pump ATPase Enzyme is inhibited and it remains as unable to be reactivated. It reduces the entry of K⁺ and exit of Na⁺. The secondary system (Na⁺—H⁺ exchanger) already mentioned is activated. According to Mahnensmith et al. 1985, this alternative secondary system plays also a pathophysiological role in diverse conditions such as cancer, renal acid-base disorders, hypertension and tissue and organ hypertrophy. Herein, it is possible to state that the Na⁺—K⁺ pump ATPase enzyme is associated to the hyperpolarization condition of normal cells and the secondary antiport system is correlated to the depolarization condition of cancer cells.

Electrical changes such as, lower EPD in rapidly proliferating and transformed cells have been reported by Binggeli et al. 1986; it has also been reported by Marino et al. 1994, specifically in breast cancer, and reported by Davis et al. 1987 and Goller et al. 1986 in colon cancer.

In agreement to aforesaid statements, cancer cells are not able to switch to a quiescent or resting stage, which provokes in such cells a perpetual and uncontrolled cellular proliferation.

Sustained depolarization in cancer cells can be considered a deviated variant of the general mitogenesis model of normal cells as observed in FIG. 3. ABA action is capable to abort the malignant mechanism by shifting cancer cells from a depolarized and damage configurational state to a hyperpolarized and normal configurational state.

### Modified Triple Layer Model

Presence of membrane-associated hCG molecule of cancer cells determines theoretically, according to this invention and FIGS. 4 and 5, a variation of the triple layer model. Three fundamental layers structure this model. They are:

1. A first layer (28), the innermost or surface layer, also called the Stern layer, which consists of the plasma membrane's solid surface with negative charges (30) and local positive ions (32).

2. A second layer (34) or diffuse layer defined by adsorbed negative and positive ions of relatively strongly bound (36).

3. A third layer (38) or hCG layer, consisting of SIA negative charges (40) in the hCG outer glycocalyx, plus adsorbed or repelled cations (42).

Many models have been developed which explain the behavior of a membrane. The diffuse double layer designed by Gouy (1910), the Stern model, the Gouy-Chapman-Stern-Grahame electric double layer and the triple layer model have widely been studied.

Original triple layer model in normal cells consists of a diffuse third layer with ions weakly attracted to the solid surface. This triple layer model of this invention is modified due to the presence of hCG in cancer cells, which makes tumor cells behave in an atypical fashion. It functions in cancer cells, as an electrical barrier 20 microns far from the Stern layer. There, negative charges (40) exert the important role of repelling cells of the immune system and serve apparently also as receptors.

Absorption of substances by the plasma membrane can be considered an electrostatic mechanism, where diffusing mechanisms are also involved. An hCG barrier produces an additional compartment area, which can be used by cancer cell to handle adequate ionic concentrations close to the plasma membrane and produce favorable concentration gradients toward or from the cytoplasm.

For example, low Na⁺ concentrations in first and second layers (28) and (34) are able to produce a concentration gradient between the third layer (38) and these inner layers (FIG. 5). Thus, Na⁺ can be efficiently diffused to inner layers and subsequently into the cytoplasm.

### ABA as Tool for Changing Polarization of hCG

Cations can be involved in binding to SIA. Tiralongo 2002, p. 4, mentions in the apart of biological roles of SIA that, due to negative charges, SIA are involved in binding and transport of positively charged molecules, as Ca²⁺. According to Delong et al. 1950, p. 718 and Ambrose et al. 1956, p. 576, cellular surface of cancer tissues shows a decreased Ca²⁺ content in comparison with normal tissue. Ca²⁺ deficiency has been associated to linking decreased adhesiveness and invasiveness of cancer cells.

Cations such as Ca²⁺ and Mg²⁺ are deficient in cancer tissues. Its exportation is not able to produce important changes in EPD. In malignant tissues the Na⁺—H⁺ antiport system is activated, and it regulates Na⁺ import to the cell and H⁺ export from it. Thus, Na⁺ becomes the most important cation cell inside; although K⁺ is contained in smaller amounts, cation exportation of both could change negative charges of hCG Transportation of such cations exerts drastic changes in EPD. ABA can be medically used in humans as an ion exporter, because it has a capability for moving these cations to the outside of plasma membrane. Na⁺ concentration gradients reversed toward the ECF have been found in relation to a cancer patient curative process. The efflux direction is defined by higher concentrations of Na⁺ contained in first and second layers (28) and (34) and lower concentrations contained in third layer (38), (FIG. 4).

According to clinical observations of Dr. Max Gerson, when cancer patients were responding to treatment, they lost extra Na⁺ from the body in the urine (Gerson M 1978, p. 454, Cope F. W 1978, p. 466). These observations of Dr. Gerson are clearly indicative that, an excess of Na⁺ is eliminated from patient body during the process of cancer recovery and, K⁺ is not.

According to Dr. Gerson the other part of the human body recovery process from cancer disease was replacement of excess Na⁺ by K⁺ in damaged tissues. Those results obtained and developed by Dr. Gerson during 30 years of clinical experimentation, are found correlated to thesis of Ling 1960, and, Cope 1978, about the association-induction hypothesis and tissue damage syndrome. Extruded Na⁺ and regained K⁺ are effects or consequences of a change of cancer cell configuration state, in which the malignant cell switches from a damage configuration state to a normal state. There, after malignant mechanism is aborted, Na⁺—K⁺ pump is activated and cell regains presence and preference for K⁺.

Healthy cell is associated with higher intracellular K⁺, lower intracellular Na⁺ and higher EPD, and cancer cell is associated with lower intracellular K⁺, higher intracellular Na⁺ and lower EPD (Cone 1975, cited in Haltiwanger 2003, p. 30). At this point, it is possible to suggest that, membrane potential (EPD) is the essential factor for switching from a damage configurational state to a normal state. A variation (increase) in EPD, inexorably conducts in cancer cell toward a normal configurational state.

ABA exporting both cations to the cell outside provokes a membrane hyperpolarization, which aborts mentioned profound and sustained cancer cell depolarization. K⁺ high mobility and Na⁺—K⁺ pump ATPase enzyme activation will facilitate that, K⁺ can be regained by cancer cell cytoplasm.

In agreement to Haltiwanger 2003, p. 41, some effects that are seen when membrane potential (EPD) is increased include: enhanced cellular energy production (ATP), increased oxygen uptake, changes in entry of Ca²⁺, “movement of Na⁺ out of the cell, movement of K⁺ into the cell”, changes in enzyme and biochemical activity, and changes in cellular pH.

It is believed in this invention that, ABA ion decrease will produce a hCG positive polarization, which is accomplished through an electron removal from hCG outer layer, toward cancer cell cytoplasm.

This last statement denotes that, a plasma membrane subtle electrostatic phenomenon might be exerted by ABA hormonal action.

### Electrostatic Phenomenon in Plasma Membrane, Redox Reaction, Electrical Equilibrium, Electron Depletion and Reorganization of Charges

According to a large number of researchers, total cell structure is connected through a liquid crystal protein polymer connective system continuum. This term has been used to express that, such system connects the cytoskeleton elements of the inside through cell membrane, as a total structure. Haltiwanger 2003, p. 20, reveals that, such a continuum of liquid crystal connections in cells, deeply studied by Becker 1974, Ho M W 1998, and, Oschman J. L 2000, allows electrons and photons to move in and out of cells. In his opinion, “cytoskeleton filaments” function as electronic semiconductors and fiber optic cables integrating information flow, both within the cell and with other cells. Also it is believed that, cytoskeleton proteins link the inside of cell like a system of telegraph wires terminating onto the nucleus membrane (Ho 1993, p. 94), or act as coherent molecular antennas radiating and receiving signals (Oschman J. L 2000, p. 131).

Dr. Merrill Garnett 1998, has studied for decades the charge transfer role and electrical current flow in the cell. He believes that DNA, cytoskeleton proteins and cell membranes transmit an inward and outward current. The inward current flows from the cell membrane to cell structures like mitochondria and DNA, and, the outward current flows back along liquid crystal semiconducting cytoskeleton proteins, and through the cell membrane to the extracellular matrix.

Nature of cellular electron movement in transport systems as it occurs, for example in mitochondria and chloroplasts has been well known, but outside those transport systems the mechanism is poorly understood (Stern et al. 1999, p. 368). Certain theories have been proposed. Through a total cell, electrical transmission must flow on protein surfaces. According to Adey 1988, p. 149, electrical interactions between cell membrane and weak electromagnetic fields are exerted through electrical charges located on cell surface macromolecules. Proteins and macromolecules function as semiconductors (Szent-Gyorgyi A 1978, 85, Brillouin L 1966). In addition to semi conductivity, complex crystalline structures possess properties such as photoconductivity and piezoelectricity (Becker 1974, p. 237). In proteins, at a specific level, a passage of electrons is produced through a major cytoskeleton component (actin) by which ionic currents is induced. Cytoskeleton structures can behave as electrical wires and are capable of functioning as nonlinear inhomogeneous transmission lines (Lin E et al. 1993, cited in Stern et al. 1999). Concentric and structured water surrounding proteins must also interact as dipole conducting electrical currents.

Whether Na⁺ and K⁺ are exported outside of cancer cell by ABA action; it occurs an electrical attraction by coulombic forces between cations and hCG negative charges. Such charges are found distributed along hCG filaments in extra and intracellular space. Therefore, such pairs of coupled positive and negative charges produce a small charge difference, prompting an electrical current through hCG filaments. This “transient coupling” of positive and negative charges, is the phenomenon of the same described and experimentally proven in vitro by Dr. Virginia Livingston, which she defined as the neutralization effect of the Microbic Chorionic Gonadotropin.

### Redox Reaction

Those pairs of coupled charges work like generators. In electrical generators, the current is driven by the potential difference or voltage. Thus, there is an established electrical field. In generators, the electrons are transferred from the negative pole toward the positive pole, through the conductor, which unifies both poles. Inside of generators and to complete the circuit, electrons are transferred from the positive pole toward the negative pole. This internal electron transfer produces the necessary electric energy. In hCG filaments, water is the conductor. In normal cells, the electrical current travel through a pattern of water concentric rings; in cancer cell presence of Na⁺ produces an abnormal loss of this pattern.

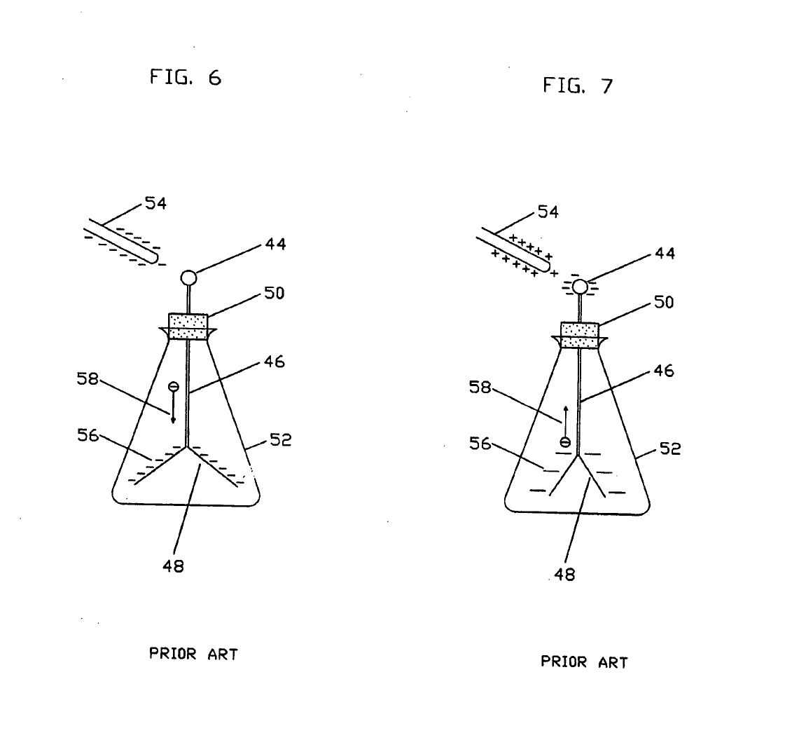

Electricity is a universal phenomenon, with physical and biological implications. So, in general sense, certain biological cellular structures have a similar behavior with electrical or electronic components. In plant and animal tissues, electricity must flow under the same concepts and laws, as it does in electrical or electronic components. In cancer tissues, when cations are exported to the cell outside and such cations confront hCG negative charges, they form ionic bonds along filaments. It produces an electrical potential difference or voltage. This mechanism drives electrons from a SIA negative charge (40) in third layer or hCG layer (38) toward the nearest ionic bond down road, where that confrontation happens. According to the electroscope device, attraction of positive and negative charges prompts an electrical current (see FIG. 7). Thus, such charge confrontation in water produces an electrolyte double redox reaction between the polyatomic ion [SIA-COO]—, which works as a cathode and the [Na]⁺ cation, which works as an anode. As water is the conductor and electrical current comes through, electrolysis is the phenomenon involved. These coupled charges immersed with water generate a electrical circuit and may be considered and are denominated redox centers:

### Cathode [SIA-COO]⁻ [Water][Na]⁺ Anode

In this case, the anode is formed because, in ABA induced cation exportation from cancer cells, Na⁺ will be the predominant cation. Likewise, in tumor cells the cathode represents the SIA negative charge which is found in the carboxyl group (See FIG. 2).

[Na]⁺ as oxidant having an electronic distribution (2,8), is reduced by winning one electron, and, [SIA-COO]⁻ as reductant is oxidized by losing two electrons. Both reactions are electronically compensated and 2e⁻ is the transferred net charge between both electrodes, as it follows:

2[Na]⁺+2e⁻→2Na (1) ANODE REDUCTION

[SIA-COO]⁻→SIA-CO⁺+½O₂+2e⁻ (2) CATHODE OXIDATION

In electrolysis, hydrogen is evolved at the cathode and oxygen is evolved at the anode, thus, water is reduced to form hydrogen and oxidized to form oxygen, as follows:

2H₂O→O₂+4H⁺+4e⁻ (3) ANODE OXIDATION

4H₂O+4e⁻→2H₂+4OH⁻ (4) CATHODE REDUCTION

Herein double redox reaction happens. In the anode, the half reactions (1) and (3) interact, and in the cathode reactions (2) and (4) do the same as follows:

2Na⁺+2H₂O→2Na+O₂+4H⁺2e⁻ (5) ANODE

[SIA-COO]⁻+4H₂O+2e⁻→SIA-CO⁺+2H₂+½O₂+4OH⁻ (6) CATHODE

In reaction (5), the anode gains two electrons from the water and loses them, which are transferred to the cathode in reaction (6). The reactions (5) and (6) combine and exchange components as follows:

2Na+O₂+4H⁺+SIA-CO⁺+2H₂+½O₂+4OH⁻→2Na+SIA-COOH+2H₂O+O₃+2H₂↑+H₃O⁺ (7)

In reaction (7) SIA-CO⁺ is reduced by one hydroxide to form the carboxyl group; 4H⁺ and 3 hydroxides form two water molecules and one H₃O⁺. Also one diatomic oxygen molecule (O₂) and one oxygen atom (½O₂) react to form ozone (O₃). The ozone in contact with water gives up its extra atom of oxygen, which is not bonded very tightly, and connects up with water to produce the reactive oxygen specie-hydrogen peroxide, as follows:

H₂O+O₃→H₂O₂+O₂↑ (8)

Final components of reaction (7) keep an acid-base ionic balance according to theory of Bronsted and Lowry (acid gives a proton and base accepts the same proton). Thus, it is established an ionic equilibrium between dissociated and non-dissociated components, according to simplified reaction (9):

2Na+SIA-COOH+H₂O+H₃O⁺⇄2[Na]⁺[SIA-COO]⁻+2H₃O⁺ (9)

Although SIA-COOH is a weak acid, complementary acidity and delocalized charge that stabilizes the conjugate base makes that acid stronger and it shifts the equilibrium to the right. Both the carboxyl acid group and the carboxylate anion (conjugate base) are stabilized by resonance, but the stabilization of the anion is much greater than that of the carboxyl group. It may also be observed in reaction (9) that one [SIA-COO]⁻ reacts with two [Na]⁺. Such characteristic might be owing to that, the negative charge of the carboxylate anion [SIA-COO]⁻ is delocalized over both oxygen atoms to form a stable resonance hybrid. Research of Hess et al. 2009, page 13299, points out that, small ions with a high surface charge density as (Ca²⁺ and Na⁺) pair up with major intracellular anions such as (phosphates and carboxylates). Contact ion pair between Ca²⁺ (with double valence) and carboxylate, must be exerted by attraction to both oxygen atoms of the anion, which shares the negative charge.

If charge confrontation happens in another redox center down road, the polyatomic ion [SIA-COO]⁻ will lose two electrons as expressed in reaction (2). It will produce a positive polarization of the hCG-membrane associated molecule and a release of [Na]⁺:

### [SIA-CO]⁺ Dissociation [Na]⁺

According to P. L Dutton et al. 1999, in the research “How Biological Molecules Move Electrons: Simplicity Trumps Complexity”, electron transfer occurs between redox centers within proteins and it is accomplished by means of an instantaneous quantum mechanical phenomenon called tunneling. Also according to Tezcan et al. 2001, electron transfer in proteins involves as a third step a dissociation of the oxidized and reduced products. Electron loss from the carboxylate anion in reaction (9) or (2) may supply a net charge of two electrons to activate another redox center. The dynamic of the process is given by an electron transfer cycle that, it will switch off a protein segment up road, and, switch on a protein segment down road. Finally, in reaction (8) one molecule of hydrogen peroxide is produced. According to Zhang et al. 2001, many metabolic processes, including chloroplastic, mitochondrial, and plasma membrane-linked electron transport systems, produce ROS such as the superoxide radical (O2⁻), the hydrogen peroxide (H₂O₂), and the hydroxyl free radical (OH⁻).

### Electrical Equilibrium, Electron Depletion and Reorganization of Charges

Energizing stages or generators will conduct the current to cell areas with bigger electron deficiency. Thus, in order the cancer cell to obtain an electrical equilibrium, the most electron probable capture area would be the cancer cell cytoplasm. According to Stern et al. 1999, malignant cell metabolism works as pumps of protons and electrons, flowing from cytoplasm and going out to increase electronegativity of the extracellular space. They also mention that, a constant flow of electrons from cytoplasm finally it causes cancer cell electron depletion.

The electron transfer directed to the cancer cell cytoplasm will increase the cell energy and EPD. These effects might reactivate the Na⁺—K⁺ ATPase enzyme, permitting it K⁺ reentry toward the cytoplasm.

Tsong T. Y 1989, and, Blank M 1987, mention that, ion accumulation near the membrane surface has been shown to reproduce some results with the Na⁺—K⁺ ATPase enzyme. It also is mentioned by Tsong T. Y in this research that, Na⁺—K⁺ pump enzyme is shown to utilize free energy transmitted through an oscillating electric field to pump Na⁺ and K⁺ against their respective concentration gradients.

Another effect produced by the electron transfer would be, the transient conversion of hCG negative charges in positive charges. When electrons are ejected to the cancer cell cytoplasm, it will produce in each pair of coupled charges in redox centers, a cation reject and ion movement by coulombic forces along hCG filaments. Polarized hCG positive charges cannot longer attach cations. It will conduct Na⁺ excess to the bulk and K⁺ returning to the cell. The end of this chain reaction, produced by ABA, may be found in connection to theory of Dr. Max Gerson.

As K⁺ returns to the cell, a change of configurational state occurs, from the damage to the normal. Likewise, as cancer cell becomes in normal, hCG is inhibited and cell recovers its original negative charge. In conclusion, during a process of cell normalization or differentiation, what it accounts is just an electronegativity reduction of the cell surface charge. The master thesis of M. E Kruse, made in 2006, reported a reduced luminescence (indicator of reduced negative surface charge), by ABA effect in four humans cancer cell lines. She assumed and attributed it to, a reduced cancer cell proliferation.

The electroscope is a device, which transmits electrical currents such as it could occur through cell filaments and structured water.

### The Electroscope Device

Haltiwanger 2003, p. 6, has cited that, there are multiple structures in the cell acting as electronic components due to, biological tissue and components can receive, transduce and transmit electric, acoustic, magnetic, mechanical and thermal vibrations. For example, membrane proteins and DNA consist of helical coils. These structures function as electrical inductors (Haltiwanger 2003, p. 5). Cell membrane functions as a capacitor with leaky dielectric characteristics (Garrison W, 1969).

Nobel prize winner Szent-Gyorgi A, resembled cell membranes as closely analogous to the PN junction, a semiconductor device used in solar cells, which facilitates a positive and negative charge separation and is capable of generating an electric current when excited by heat or light (Ho 1993, p. 102).

The electroscope device, which determines or measures presence of electrostatic forces has a strong correlation to the electron transfer theory in liquid crystal continuum system of the cell. Two different directions follow up the electron movement across the plasma membrane. One direction is induced by the phenomenon of ion decrease, and another opposite direction is induced by the phenomenon of ion absorption. It has already been above mentioned, the generated mechanism of ABA “ion decrease” in cancer cells. If one cation as Na⁺ or K⁺ is exported outside of plasma membrane, one or undefined amount of electrons comes back to the cell in order to restore both, the cell electrical equilibrium and the cytoplasm electron depletion. Nevertheless, in ion absorption phenomenon the electron transfer direction is complete opposite.

In graphic 6 and 7 of this invention, it can be observed two identical electroscopes in contact with negative and positive charged bodies. Several elements of the electroscope device have a similar element in normal and cancer cell plasma membrane, according to the following similes: the metal ball (44) can be considered SIA receptors showing plasma membrane negative electric charges in inner and outer layers. The metal rod (46) can be considered glycoprotein extended filaments or projections coming from the membrane-associated hCG molecule. The metal leaves (48), likewise, can be considered the plasma membrane and Na⁺ channels. The negative and positive charged bodies of the electroscope can be considered ions as, for example Na⁺ (54). The electron (56) and the electron transfer (58) have no simile, because they are naturally identical. The electroscope glass container (52) and gasket (50) can be considered the plasma membrane lipid bilayer isolating properties. In FIG. 6, if it is approached to the metal ball (44), a negatively charged body (54), for example a frictionally resin rod, some electrons (56) are transferred (58) through the metal rod (46) toward the extreme of the device, where the metal leaves (48) are. Such metal leaves (48) will widely open due to coulombic forces, which repel charges of the same polarity. In FIG. 7, if it is approached to said metal ball (44), a positively charged body (54), said metal leaves (48) get closer, due to that electrons (56) are transferred (58) to the metal ball (44). Likewise, if this metal ball (44) comes in contact with the ground, for example, touching it with a finger, some electrons (56) will escape from the electroscope glass container (52).

In malignant tissues, Na⁺ absorption has been found correlated to electron efflux. According to Stern et al. 1999, page 368, trans plasma membrane electron transport is coupled to the Na⁺—H⁺ antiport exchange, and electron efflux is associated with proton efflux. It means that, when one ion Na⁺ is transported to the intracellular space by the antiport system, one proton and one electron or undefined amount of those is exported to the extracellular space. In normal cells as in cancer cells as well, when a current of cations gets close to the plasma membrane in order to be absorbed, the electron transfer nature follows a stimulated electrical current in the electroscope device as shown in FIG. 7. According to it, if a positively charged body (cation) approaches to SIA negatives charges of proteins, electrons escape from cell cytoplasm and plasma membrane, causing it a depolarization. The following statements explain in detail, these electron transfer phenomenon implications.

### Depolarization of Plasma Membrane Caused by Ionic Currents

A model developed by Tsong 1989, has postulated that, a protein can undergo conformational changes by a coulombic interaction with an oscillating electric field.

It is possible to assume that, cation attraction to negative charges of sialoglycoproteins, in the plasma membrane and ion channels, can generate electrical changes in such proteins.

Study of the electroscope device can suggest that, electron transfer may also occur from the plasma membrane, to currents of cations causing a removal of negative charges (electrons) in cell membrane and ion channels. When said electrons are removed, conformational changes happen in the plasma membrane, most importantly a depolarization.

According to Bennett et al. 1997, in an article titled “Contribution of Sialic Acid to the Voltage Dependence of Sodium Channel Gating. A Possible Electrostatic Mechanism”, changes in Na⁺ ion channel of rat skeletal muscle were observed, after enzymatic action of neuramidase, such as SIA removal. These changes in channels were, 10 mV more depolarized than control channels. Bennett et al. 1997 also point out that, a general feature of many Na⁺ ion channels is that they are heavily glycosylated with a large carbohydrate fraction in the form of SIA. In this mentioned research of Bennet et al. 1997, SIA negative charges removed from ion channel surfaces is found associated to depolarization.

### Electrostatic Phenomenon Hypothesis in Na⁺ Channel.

In aforesaid statements it can be observed that, an interaction between cation currents and Na channel SIA negative charges, may be given through an electrostatic phenomenon.

From a general knowledge, a plasma membrane depolarization effect probably produced by electron removal has a consequence in opening ion channels.

Opening and closing ion channels can be interpreted in this invention, through the electroscope device, to eventually demonstrate that electron transfer is the underlying mechanism in plasma membrane ionic interactions.

According to Marban et al. 1998, p. 647, Na⁺ channels consist of various subunits, but only the principal (alfa) is required for function. The alfa subunit has a modular architecture: it consists of four internally homologous domains (labeled I-IV), each of which contains six trans membrane segments. The four domains fold together so as to create a central pore. According to Stuhmer et al. 1989, the fourth trans membrane segment (S4) stereotypically studded with positively charged residues, lies within the membrane field and moves in response to depolarization, somehow opening the channel. In agreement to Bennet et al. 1997, p. 327, the segment (S4) consists of a repeated triad of two hydrophobic amino acids followed by a positively charged residue, consistent with a role as a voltage sensor, residing within the membrane bilayer. The rest of the segments of the Na⁺ channel are negatively charged with SIA. Schematic depictions of the Na⁺ channel alfa subunit are shown by Marban et al. 1998, p. 648.

According to Catterall, W. A 1992, it is assumed a helix model of each positively charged residue in the (S4) segment. In it, each positive charge is paired with some negative charge on the adjacent S1 to S6 segments (cited in Aidley et al 1996, p. 186). This model appropriately suggests that, coulombic interactions (attraction and repulsion forces) occur between paired negative and positive charges in channels.

On the other hand, Hodgkin and Huxley 1952, found that changes in ionic permeability were associated with the movement of some electrically charged particles within the membrane. Thus, movement of charged particles may be given toward positive charges of segment (S4) or from negative charges of adjacent segments of channels.

Stuhmer et al. 1989 also found that the steepness of the relation between channel opening and the membrane potential was progressively “reduced” as the positively charged residues of the (S4) segment were replaced by neutral or negatively charged residues. This research, automatically realizes that, movement of charged particles mentioned by Hodgkin and Huxley 1952, happens from negative charges of adjacent segments of channels.

In this invention, it is disclosed under the electroscope device standpoint that, cation currents close to the channel will produce a electron removal from the negatively charged segments of the channel, decreasing negative charges in such segments or increasing positive charges in it. At that point, a positive charge is generally expressed by all the segments. When it occurs, segment (S4) is moved away from the rest of the segments due to repelling coulombic forces. Removed electrons from the channel, neutralize said current of cations. Thus, such ions switch from an ionic stage to a neutral or uncharged stage (non-polar). When neutral particles get across the channel, negatives charges acquired before entering to the pore are incorporated back to the channel. After the process is completed, and once electrons get back to adjacent segments during the passage, voltage sensor positive segment (S4) is again attracted to negative segments of the pore. This latter phenomenon produces the closing of ion channel.

The cell membrane lipid structure, makes it relatively impermeable to the passage of charged molecules. This well-known lipid membrane transport property can be understood as a generalized rule, if charged molecules or ions get across channels as uncharged molecules, according to theoretical gating model expressed in this invention.

### Apoptosis by ABA Action

According to Fingrut et al. 2002, from the Tel-Aviv University, plant stress hormones as sodium salicylate (SA), jasmonic acid (JA) and methyl jasmonate (MJ) can suppress the proliferation or cause apoptosis in certain mammalian cancer cells (lymphoblastic leukemia, prostate, breast and melanoma human cancer cells). Although SA, JA and MJ rather hold a secondary role as plant inhibitors, this evidence reveals the power of plant stress hormones against cancer. In this invention, ABA has been correlated to a process of cancer cell normalization. Nevertheless and paradoxically, an apoptosis phenomenon is induced by ABA hormonal action as well. It was experimentally confirmed for first time in cancer cells by Hong et al. 2006, from the Chengdu Biological Institute Academy of Sciences and by Marianne Ehrhorn Kruse, then at the University of Southern Denmark.

In plants and according to Vanyushin B. F et al. 2004, peroxides, ABA, ethylene releaser ethrel, and DNA methylation inhibitor 5-azacytidine induce and stimulate apoptosis. This research points out distinct ultra-structural features of apoptosis such as: compaction, vacuolization and fragmentation of cytoplasm in the apoptotic cell; appearance in the vacuole of unique single-membrane vesicles containing active organelles; cessation of nuclear DNA synthesis, and, condensation and margination of chromatin in the nucleus; internucleosomal fragmentation of nuclear DNA; and intensive synthesis of mitochondrial DNA in vacuolar vesicles. According to Hong et al. 2006, from The Chengdu Institute of Biology, ABA produced changes in morphology of cancer cells in DU-145 (prostate cancer) and in HL-60 (promyelocytic leukemia). The results showed: in DU-145 tumor cells (growth in poor condition and nuclear pycnosis) and in HL-60 tumor cells (absence of tumor cell nuclear membrane microvillus and presentation of typical apoptosis characteristics). It was also found in both types of cancer cells that, tonofibril disappeared and number of cell organs was reduced.

It has been held in this invention that, ABA produces unequivocally cancer cell ion decrease. In a cell normalization process caused by ABA, K⁺ is regained in order to: normalize cell condition by incorporating lost water, re-establishing charges in proteins and changing the metabolic condition. Nevertheless, during an ABA apoptotic process, K⁺ uptake is inhibited being it not regained by cancer cell. This phenomenon may be found in connection to production of peroxides during apoptosis. Zhang et al. 2001 demonstrated that potassium channels are inhibited by hydrogen peroxide mediate ABA signaling in Vicia guard cells. It may be also suggested that ROS and peroxides interfere with the re-activation of the Na⁺—K⁺ pump. This phenomenon would be part of a cellular process of destruction and fragmentation.

Ion decrease from cell produces an intracellular water reduction, because Na⁺ and K⁺ carry a lot of water molecules. It reduces volume and size in apoptotic cancer cell, and probably also in apoptotic senescent normal cells.

This phenomenon of Na⁺ and K⁺ decrease has been found in connection to a mechanism of cell shrinkage in apoptotic cells (Bortner C D et al. 1997, 1998; Gomez-Angelats et. al 2000; Mann C. L 2001; Nukui M et al. 2006).

Specifically and according to Bortner C. D et al. 1997, “K⁺ and Na⁺ Efflux” play a primary role in apoptosis activation. In 1998, Bortner C. D et al. also mention that, loss of cell volume had been thought to be a passive secondary feature of apoptosis, but it has now become an area of research interest. Likewise, in agreement to Gomez-Angelats et al. 2000, this knowledge may also have an impact on the design of therapeutic strategies for a variety of diseases of the immune system in which apoptosis plays a central role, such as oncogenic processes or immune system disorders.

Although the involved apoptosis signaling molecule has not yet been identified by medical physiologists, ABA could be casually related in producing apoptosis and also the “Efflux of Na⁺ and K⁺”. This cell shrinkage phenomenon, which occurs during induced ABA apoptosis, has technical similarities with the plant stomata closure mechanism. In stomata movements, the solutes essentially efflux back and forth across the plasma membrane, therefore, the phenomenon can be turned and reversed depending the plant hydration condition. Apoptosis is not a reversible phenomenon.

### ABA in Relation to the Immune System

ABA K⁺ and Na⁺ efflux from cell is apparently a common phenomenon during ABA cell normalization and apoptosis. ABA cell normalization produces that, K⁺ and water return to the cancer cell cytoplasm, transforming the cancer damage condition toward a stage of normal. When the cell gets through an apoptosis process, it is produced K⁺ uptake inhibition. If Na⁺ and K⁺ uptake is inhibited, the cell shrinks in an irreversible way. It produces the cell water to get out provoking cell volume loss or cell shrinkage.

Whether water and ions have been lost, shrinking cell and fragments would turn toward a permanent positive polarization of membrane-hCG associated molecule. This phenomenon may stimulate an attraction between apoptotic cell or its fragments and immune system cells. K⁺ and Na⁺ uptake inhibition by cell produces respectively electron inhibition efflux toward the extracellular space. Thus, cancer membrane cell positive charge remains unaltered. This well designed and intended mechanism by nature will produce the necessary attraction, between cancer and immune cells. Otherwise, those cells would remain immunologically inert. New investigations about ABA could suggest the real function of ABA as an endogenous cytokine, once that human immune system cells target cancer cells for attacking and destruction.

According to Bruzzone et al. 2007, ABA has been identified as an endogenous cytokine in human granulocytes. They mention that, ABA stimulates several functional activities as phagocytosis, reactive oxygen species (ROS) and nitric oxide production, and chemotaxis of human granulocytes. In agreement to them, increase of free intracellular ABA and its release by activated human granulocytes indicate that, ABA should be considered as a new pro-inflammatory cytokine in humans. In addition, invention of Zocchi et al. 2008, titled “Fluridone as an Anti-inflammatory Agent” teaches that, by using HPLC-MS analysis, ABA presence was demonstrated also in human lymphocytes, fibroblasts, mesenchymal stem cells (in bone marrow stroma precursors), platelets and monocytes.

It has been stated in this invention, that equal polarity of hCG in cancer cells and immune system cells make such cells immunologically inert and unable to get close and attack tumor cells (Acevedo H 2002, p. 136). Because theory of Acevedo H is highly probable and correct, only cancer cells expressing an membrane positive surface charge might be attracted to immune system cells, which express negative surface charge.

Considering the investigations of Bruzzone et al. 2007 and invention of Zocchi et al. 2008, it may be suggested, that ABA must be used by immune cells for producing a sequential steps of events. Changing the polarity of hCG-membrane associated molecule must be a prior step of immune cells to get close to cancer cells for phagocytosis and destruction. In addition, ABA also stimulates production of other cytokines as tumor necrosis factor alfa (TNFALFA) and prostaglandins E₂ (PGE₂). Invention of Zocchi et al. 2008, demonstrates that, among other cytokines, TNFALFA is increased by ABA effect specifically in human monocytes, murine microglia and murine macrophages. Cells commit suicide by apoptosis when it triggers death activators as TNFALFA, TNFBETA and FAS ligand binding to receptors at the cell surface.

Research of Scarfi S and Zocchi E, and collaborators, on 2008, titled “Cyclic ADP-Ribose-Mediated Expansion and Stimulation of Human mesenchymal stem cells (MSC) by the Plant Hormone Abscisic Acid”, mentions that ABA stimulates functional activities of MSC: cyclooxygenase 2-catalized production of PGE₂, release of several cytokines known to mediate the trophic and immunomodulatory properties of MSC, and chemokinesis.

Human immune system cells function as a protective barrier against cancer cells and pathogens. Thus, it is possible to express that, the immune system has been trained through the evolution to offer resistance, recognize and attack cancer cells. The human immune system definitively destroys cancer cells in healthy metabolisms, when such cells rise and start to grow. Such theory has been supported since 1908, by the German Nobel Prize Winner P. Ehrlich. He postulated that, cancer occurs spontaneously in vivo and the immune system is able to both, recognize and protect against it. During the last century, there has been a long running controversy about the hypothesis that, the immune system is not a player in a cancer resistance, control and destruction. Nevertheless, it was demonstrated that, immune cells represent a major barrier to cancer progression. The best tool to demonstrate the phenomenon came out with the important work of Robert Schreiber's Lab, showing that, immunodeficient animals (RAG− knockout mice) are more susceptible to tumors. After the discovery of the Acquired Immunodeficiency Syndrome (AIDS), similar evidence also came out to explain the above mentioned phenomenon. The Kaposi Sarcoma is a type of cancer, which develops when the immune system weakens in patients with AIDS. Robert Schreiber's research defines that, cancer cells develop an interaction, which takes three forms (Elimination, Equilibrium and Escape). Equilibrium between cancer and immune cells is maintained through an adaptive immunity.

In 1957, Burnet and Thomas proposed that, lymphocytes act as sentinels in recognizing and eliminating continuously arising, nascent transformed cells. According to the immunoediting process of elimination, defined by R. Schreiber, initiation of an anti-tumor immune response induces inflammatory signals and provokes a recruitment of cells of the innate immune system. In this phase, infiltrating lymphocytes are stimulated to produce interferon (IFN-gamma). According to H. Ohtani (2007), tumor infiltrating lymphocytes are quite significant in human colorectal cancer.

When released, IFN-gamma increases recognition of tumor cells by up-regulating antigen presentation to T-lymphocytes. During this process, macrophages, dendritic cells and other cell types capture antigens, and it enables their recognition by T-cells. Thus, during the second phase of elimination, Schreiber mentions that, newly synthesized IFN-gamma induces tumor death promoting the production of chemokines (CXCL10, CXCL9 and CXCL11). It herein must be suggested that, the lately role of IFN-gamma, in this second phase of elimination, must be submitted to further investigation. Whether, considered, ABA discovery performed by S. Bruzzone and E. Zocchi and associates (2007), a review of the cancer immunoediting process of R. Schreiber is required; it in order to be open to some new hypothesis and ideas, which must investigate a probable cross talk signaling induction between T cells and immune cells, as granulocytes and other cell types. Such phenomenon could be produced to stimulate ABA absorption, immune cell activation and posterior use of ABA as a cytokine. It would allow immune cells to produce an attack and destruction of cancer cells, otherwise cell normalization.

Cancer cell recognition always might be executed without limitations, because IFN-gamma can internally be produced and segregated by immune system cells. Nevertheless, a triggering of a mechanism of cancer cell attack can be blocked showing a restrictive response. The reason of that has been set in this invention, ABA is not produced by immune cells, it is absorbed from blood at determined biological active concentrations. That means, immune cells do not have limitations in using IFN-gamma for recognition, however they do in using ABA to attack cancer cells. It helps to explain the scientific fact that, immune cells are occasionally found in tumors, and sometimes they do not attack cancer cells. When this phenomenon occurs, it is mistakenly expressed that, immune cells were not able to recognize a determined specific cancer antigen.

Correlation of the ideas mentioned above could open new lines of investigations about ABA, in association to healthy and cancer sick metabolic conditions and in association to the human immune system.

Investigations of Bruzzone et al. 2007 and Zocchi et al. 2008 may be considered significant, and additional evidence going forward, after Robert Schreiber's research that, immune system is important for control, destruction and normalization of cancer cells. Therefore, any prior medical treatment, which it compromises the patient immune system, such as chemotherapy or radiotherapy could affect a subsequent ABA treatment or any treatment against cancer. Also, cancer patients that have received prior conventional treatments such as chemotherapy or patients with immune system disorders must be refused to participate in an ABA clinical trial, before it could be demonstrated ABA efficiency in the treatment of cancer without the involvement of the immune system in such treatment.

### ABA Expression of Retinoic Acid Receptor Beta, PPAR Gamma, Involucrin Protein and Inhibition of Ki67 Cancer Marker

For long time, it has been recognized retinoic acid (RA) as a signaling molecule influencing developmental processes and cell differentiation. Since 1950, many investigations had proven its limited power as differentiation inducer drug. Nevertheless, it was reported by Khuri et al. 2006, during the largest retinoid chemoprevention trial that, retinoid (ISOTRETINOIN) was not effective against head and neck squamous cell carcinoma.

According to Freemantle et al. 2006, in an article appeared in the editorials of the Journal of The National Cancer Institute vol 98, No 7, it has been discovered that, retinoic acid receptor beta (RARBETA) expression is frequently silenced in epithelial carcinogenesis. It has also led to the hypothesis that, RARBETA acts as a tumor suppressor and is partially responsible for the limited clinical activity of classical retinoids.

Herein, it is important to mention that, under a treatment with ABA, RARBETA is expressed and not silenced by cancer cell. The expression of this receptor has been confirmed by Zhao et al. 2007, in the Key Laboratory of Oral Biomedical Engineering of Ministry of Education, in Sichuan University. The research titled “Effect on Induction of Differentiation of TCA8113 Cells Affected by Abscisic Acid in Vitro”, confirms the expression of RARBETA, involucrin protein and caspase-3mRNA.

According to Donato and Noy 2005, presence of RA is not necessary for over expression of the cellular retinoic acid binding protein II (CRABP II), which is the key protein attaching to RARBETA. Also, they mention that the tumor suppression by RA regulates transcription of multiple genes.

Investigations mentioned above prove that, it is ABA and not RA, which induces and conducts efficiently a process of cell differentiation.

According to Freemantle et al. 2006, apparently retinoids activate transcription by binding to the classical nuclear retinoid acid receptors (RARS) Alfa, Beta and Gamma and also to the non-classical nuclear retinoid x receptors (RXRS). RARS can heterodimerize with RXRS, whereas RXRS heterodimerize with other nuclear receptors, including the thyroid hormone receptor, the vitamin D receptor, hormone receptor, the vitamin D receptor, and the peroxisome proliferator-activated receptors (PPAR).

The invention of Bassaganya-Riera et al. 2007, Number WO2007/092556A2, titled “Method of Using ABA to Treat and Prevent Diseases and Disorders”, teaches that PPAR gamma forms a heterodimer with RXR and undergoes a conformational change that allows it to recruit coactivators. The primary outcome of the PPAR-controlled transcriptional regulation of genes is a reduction in the hyperlipidemia, hyperglycemia, and hyperinsulinemia. The invention is based in that, ABA can affect the expression of PPAR Gamma and relates to prevention and/or treatment of hyperglycemia, impaired glucose tolerance, insulin resistance, prediabetes, and Type 2 diabetes, while in other embodiments, the invention relates to prevention and/or treatment of inflammation, including but not limited to obesity-related inflammation.

ABA capacity in diabetes treatment is also disclosed in an invention of Bruzzone S et al. 2008, from the Department of Experimental Medicine, Section of Biochemistry and Center of Excellence for Biomedical Research, University of Genova. The invention titled “ABA is an Endogenous Stimulator of Insulin Release from Human Pancreatic Islets with Cyclic ADP Ribose as Second Messenger” teaches that, ABA is an endogenous stimulator of insulin secretion in human and murine pancreatic beta cells. They mention that, autocrine release of ABA by glucose-stimulated pancreatic beta cells, and the paracrine production of the hormone by activated granulocytes and monocytes suggests that, ABA may be involved in the physiology of insulin release as well as in its deregulation under conditions of inflammation.

Moreover, ABA produces additional changes in cancer cell. According to Zhao et al. 2007, the Involucrin protein is expressed during a cancer cell differentiation process of TCA8113 (human oral carcinoma), affected by ABA. In connection to this, other researchers have found that, involucrin protein is expressed when stem cells from basal layers of the human skin are differentiated to form keratinocytes in upper layers. On the other hand, and in agreement to Hong et al. 1996, the nuclear protein Ki67 that is expressed in proliferating tumor cells was reduced by ABA, which decreased such proliferating activity. Ki67 has been used as a cancer marker for cell proliferation of solid tumors and some hematological malignancies.

### ABA Involvement in Animal Apoptosis of Senescent Normal Cells

In this invention, it has been suggested ABA role intervention in cell normalization and differentiation processes. Nevertheless, ABA could be implicated in aging and cellular death processes as well. The following several reasons provide evidence that, ABA could hypothetically be involved in apoptosis of senescent normal animal cell: 1. ABA and ethylene have been correlated to the phenomenon of senescence and plant death. 2. K⁺ and Na⁺ decrease effect induced by ABA is exerted without a criterion of selectivity. It suggests that ABA would cause the decrease of Na⁺ and small amounts of K⁺ in cancer cells and would either cause the decrease of K⁺ and small amounts of Na⁺ in senescent normal cells. 3. Apoptosis of “normal and cancer cells” has been connected to the phenomenon of Na⁺ and K⁺ decrease and cell shrinkage. 4. Plant stress hormones suppress the proliferation and induce apoptosis in human cancer cells.

### ABA Against Human Stress

ABA is considered a hormone biologically designed by nature to defend plants against stress. ABA enhances plant adaptation to various stresses such as cold tolerance, salt osmotic adjustment and drought (Zeevaart et al. 1988). In addition, ABA-glucose ester (physiologically inactive form of ABA) accumulates in plant tissues with the age and during stress treatments (Dietz et al. 2000). Those considerations could open new insights and investigations concerning ABA.

ABA plant stress adaptation could have been transferred to animals during the evolution, under the form of defense mechanisms against physical, mental and emotional stress. Mechanisms such as ABA cellular apoptosis and aging could be the ending consequences of human stress assimilation. ABA anti-stress mechanisms could take place, because ABA release of acute stress could have an antagonistic action and interactive effect with adrenalin and glucorticoids, both hormones released by the adrenal glands.

During stress, elevated levels of the human stress hormone cortisol counteract insulin by increasing gluconeogenesis. Cortisol also slows the production of good prostaglandins, increases hyperglycemia and blood pressure, and weakens the activity of the immune system. ABA opposite action, helps to release insulin from pancreatic islets (Bruzzone S et al. 2008), reduces hyperglycemia (J. Bassaganya-Riera et al. 2007), stimulates release of PGE₂ by MSC (Scarfi et al. 2008), and strengths the immune system by stimulating granulocytes and other immune system cells (Bruzzone et al. 2007, Zocchi et al. 2008). Also, good PGE₂ supports immune function, dilates blood vessels, inhibits thick blood and are anti-inflammatory.

### ABA G-Protein Signalling Pathway

According to Kennedy B-K 2003, from Penn State University, plants respond to environmental stresses with a sequence of molecular signals known in humans and other mammals as the G-protein signaling pathway.

In human cells, this mechanism has been recognized as responsible in regulating either the opening of ion channels and activities of intracellular enzymes. Coursol et al. 2003, p. 651, in the research “Sphingolipid signaling in Arabidopsis Guard Cells Involves Heterotrimeric G Proteins”, showed that a metabolite denominated, sphingosine-1-phosphate (SIP), functions in animals as an intracellular messenger and an extracellular ligand for G-proteins-coupled receptors of the receptor family, regulating diverse biological processes. In this research it was discovered in Arabidopsis that, SIP is a signaling molecule involved in ABA regulation of guard cell turgor. It also was reported that, an enzyme responsible for SIP production, sphingosine kinase (SPHK), is activated by ABA in Arabidopsis thaliana and is involved in both, inhibition of stomata opening and promotion of stomata closure.

In human cells, ABA G-protein signaling pathway has been confirmed. ABA stimulates several functional activities in human granulocytes (Bruzzone et al. 2007, page 5759) and stimulates insulin release in human pancreatic islets (Bruzzone et al. 2008, page 32188). Both researches point out that, ABA effect is produced through an identical signaling pathway sequentially involving a pertussis toxin (PTX)-sensitive G protein/receptor, protein kinase A activation, ADP-Ribosyl cyclase phosphorylation, and consequent cyclic-ADP ribose over-production, leading to an increase of the intracellular Ca²⁺ concentration.

Several studies have demonstrated that, ABA effect on drought, cold and high salt, results in Ca²⁺ levels rapid increase in plant cells (Tuteja 2007, page 136). Correlation of studies of Bruzzone et al. 2007, 2008 and Tuteja 2007, reveals that ABA signaling Ca²⁺ increase is a similar and paralleled mechanism, which it occurs in plants as in animals as well.

ABA Ca²⁺ influx and K⁺ efflux are mechanisms sequentially connected, in ABA transduction. According to Schroeder et al. 2001, in guard cells, ABA induces cytosolic Ca²⁺ elevations, which activates anion release. This causes guard cell depolarization, which activates outward-rectifying K⁺ channels, resulting in K⁺ efflux and stomata closure. K⁺ efflux finally causes membrane hyperpolarization. A large percent of drugs approved for use in humans target the G-proteins signaling pathway.

Preparation of the Medication. Curve of Uptake Efficiency of ABA

Understanding how ABA works in plants could open new insights for cancer treatments and applications. Stomata pH changes generated by light have been thought to occur in relation to photosynthesis. A CO₂ concentration reduction in guard cells as a result of photosynthesis consumption causes a pH rise. During the darkness, photosynthesis stops, and CO₂ concentration rises as a result of respiration (Devlin, 1966, p. 72). The latter phenomenon produces a pH decrease according to photosynthesis reaction linked to carbonic acid (H₂CO₃) equilibrium:

6H₂O+6CO₂^{→CHLOROPHYLL→}C₆H₁₂O₆+6O₂↑[PHOTOSYNTHESIS]

H⁺+HCO₃⁻⇄H₂CO₃⇄H₂O+CO₂ [↑H₂CO₃ ↓pH]

ABA-GE(_{INACTIVE})⇄AtBG1(_{β-GLUCOSIDASE})⇄ABA⁻(_{BIOACTIVE})+β-D-GLUCOSE [ABA HYDROLYSIS]

(_{CYTOPLASM})ABA⁻+H⁺⇄ABAH⇄ABA⁻+H⁺(_{APOPLAST}) [ABA RELEASE]

Mesophyll and epidermis cells of leaves also intervene in the stomata response. Such cells are able to store ABA when intracellular pH is relatively high during the day, but at night intracellular pH decreases and ABA is released outside plasma membrane, activating it stomata closure. This daily cycle and pH changes can be understood through the ion trapping concept of ABA.

ABA is a weak acid, which preferentially accumulates in the more alkaline compartments of the leaf. At an acidic pH (5.2 to 6.5) more ABA will be present in its lipophilic not dissociated form (ABAH). Such form can diffuse across the plasma membrane into the more alkaline compartments of the cytoplasm. In such mesophyll and epidermis cell compartments, which have a pH between 7.2 and 7.4, it dissociates to lipophobic form (ABA⁻ and H⁺), which becomes trapped inside the cell (Hartung and Slovik 1991).

ABA physiological behavior, in relation to an extracellular pH in plant cells, will be taken as reference for choosing the right pH for a buffer solution. It will permit to figure out consequentially, a pH of the medication to get the best ABA treatment against human cancer cells. This medicine has not before been applied in humans by intravenous way; therefore ABA plant physiology is the unique available reference.

An ABA efficient response in plants can be evaluated by its capacity to inhibit stomata opening or stimulating stomata closure. ABA concentration applied via extracellular, and, medium pH are important factors. Anderson 1994, p. 1177, found that 10 mcM ABA extracellular application, inhibited stomata opening by 98% at pH 6.15 and by 57% at pH 8.0. In this same research, he also mentioned that, a pH dependence of extracellular ABA action might suggest a contribution of an intracellular ABA receptor in stomata regulation. In agreement to Allan et al. 1994, p. 1107, ABA on guard cells is more effective at pH 5.5, than at pH 7. Other several researchers also found that ABA was much more effective in closing stomata pores at an acidic extracellular pH (Ogunkanmi et al. 1973, Kondo et al. 1980, Kondo and Maruta 1987, Paterson et al. 1988). If the extracellular medium pH ranges between 5.2 and 6.5, ABA produces stomata closure at first contact and signal, but also at this pH, the molecule rapidly gets across the plasma membrane (Hartung and Slovik 1991). Thus, the hormone gets in contact with intracellular receptors, by which it starts to open stomata. According to Ilan et al. 1994, an extracellular pH reduction from 8.1 to 5.5 significantly reduced outward K⁺ currents. Likewise, Blatt 1992, found that acidic extracellular pH activated inward K⁺ channels in Vicia faba guard cells (cited in Wilkinson and Davis 1997, page 569). Specifically the last two researches describe up a sequential and combined phenomenon, where the plant hormone ABA, produces: first a stomata closure and solute exit, and, second a stomata opening and solute reentry. This phenomenon links ABA to a combined phenomenon of stomata closure and opening in connection to an acidic extracellular pH range. Ahead, the ABA effect in an alkaline extracellular medium is examined.

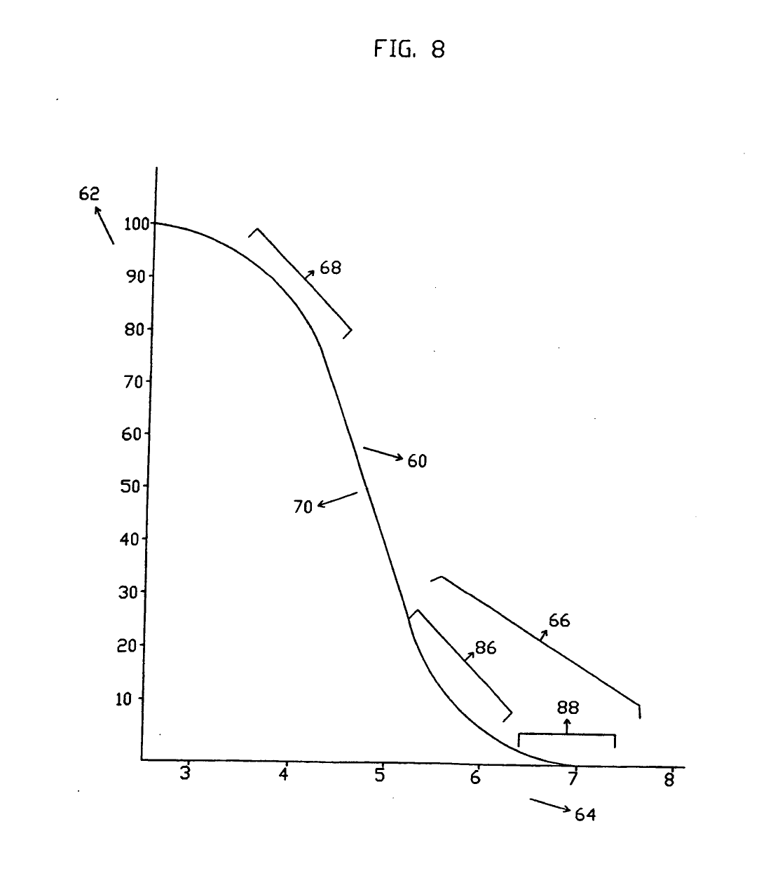

Astle et al. 1980, detected an ABA carrier restricted to root apical tissue that, was dependent on a membrane pH gradient, but not on a membrane electrical gradient. According to Wilkinson and Davis 1997, pp. 571-572, this carrier was responsible for a portion of the ABA taken up by epidermal symplast at an extracellular pH 6.0. They mentioned that both, carrier-mediated and the diffusive uptake contribute to the efficiency of ABA sequestration by this tissue at extracellular pH 6.0. Mostly important and in agreement to them, the carrier becomes inactive at an extracellular pH 7.0, when diffusive uptake is reduced and ABA accumulates in the apoplastic compartment in the intact leaf. This investigation discloses that, ABA carrier is inhibited at pH 7.0 and higher. Thus, the plant hormone is not transported toward cell cytoplasm, by which is unable to get in contact with intracellular receptors for producing stomata opening. If pH of the extracellular medium is held at a pH between 7.2 and 7.4, ABA is not able to diffuse across the plasma membrane because it dissociates to ABA-lipophobic form (Hartung and Slovik 1991). It can be observed in the FIG. 8 of the present invention that, ABA uptake is zero at an extracellular pH 6.5 and higher. At pH ranging between 5.2 and 6.5 the molecule tends to produce a combined effect of stomata closure and opening. This effect produces exit and reentry of cations. In an alkaline medium pH, ranging between 6.5 and 7.4, ABA tends to induce just stomata closure and exit of cations.

ABA uptake must be measured as function of the following variables: stomata closure and opening, the extracellular medium pH, plasma membrane inside and outside receptors, and saturated ABA uptake component (carrier). ABA taken up or not will determine, consequent hormone effect in producing cancer cell normalization or apoptosis.

Hornberg et al. 1984, pp. 321-323, found occurrence of a high-affinity guard cell specific ABA-binding proteins facing the apoplasmic space. Most importantly they detected two types of ABA receptor designated sites on plasma membrane.

Microinjections of ABA into guard cells did not inhibit stomata opening (Anderson 1994, p. 1182, and, Popova 2000, p. 379). Such data mostly provides evidence that, a reception site for ABA is on an extracellular side of the plasma membrane, nevertheless, research of Pedron et al. 1998, p. 390, agrees with a dual location of ABA reception sites (intracellular and out-facing plasma membrane). They express that, presence of intracellular receptors is in agreement to evidence that, cells also respond to intracellular ABA.

An ABA uptake theoretical curve (60) correlates ABA uptake concentrations in percents (62) and pH values of a medium outside the plasma membrane (64). It has been drawn in the FIG. 8, according to the Henderson-Hasselbalch equation:

pH=pK+log [conjugate base]/[conjugate acid]

As the protonated or undissociated form of ABA is ABAH, and the anion is ABA⁻, the mentioned equation results as:

pH=pK+log [ABA⁻]/[ABAH]

In the equation ABA pK has a value of 4.7 (70).

The pH values in variable (64) were calculated by considering relative concentrations in percent (62), of two different forms of ABA.

As it can be observed in the FIG. 8, (ABAH) uptake concentrations (62) increase when it decreases pH values (64), therefore, in acidic medium ABAH can easily get across plasma membrane. According to the FIG. 8, a range of pH between 5.2 and 7.4 varies to produce a differential effect for the maximum efficiency of stomata closure (66). At pH medium ranging between 5.2 and 6.5, ABA produces a phenomenon of cell normalization or differentiation (86). At pH ranging between 6.5 and 7.4 (88), ABA induces an apoptotic phenomenon. Conventional cancer treatments have been designed to control the disease via destruction of cells. ABA is classified and catalogued as a differentiation inducer drug. This group of agents has the natural property to control and fight cancer via conducting tumor cells to be reversed in normal cells. Cancer cell destruction by apoptosis is useful in determined patients, but not in all. Such treatment is not in agreement to perse ABA essential concept as differentiation inducer drug. In general, apoptosis causes cancer cell destruction and toxins unload into the blood patient. ABA buffer medication elected with the intention of inducing apoptosis brings a bigger risk to patients with terminal types of cancer. ABA buffer medications for inducing “cell normalization or apoptosis” must be differentially applied to cancer patients by physician criterion (see aside of the operation).

In addition, it is possible to observe on the curve (60), an area of stomata opening maximum efficiency (68), which is produced at a pH 5.2 and lower. At this point, ABA absorption has been completed in order to full maintain the phenomenon of stomata opening.

### Concentrations and Medication Dosage

In survey of Hartwell 1982, an enormous quantity of plant species has been found in showing properties against different types of cancer, generating it perceptible benefits. In plants, ABA concentration is considered relatively very low; notwithstanding ABA is the common factor found in all species of plants. ABA concentrations in unstressed leaves range approximately between 800-1500 ng/100 g of fresh weight, meanwhile, in water stressed leaves ABA ranges between 1700-10000 ng/100 g of fresh weight (Singh et al. 1979, p. 136). According to this research, ABA levels increased due to water deficit by at least in an average of 2-7 fold. Other researchers mention that ABA level increased in about 30 and even 200 fold. In xylem sap of well watered sunflower plants, ABA concentration ranges between 1.0 and 15.0 nmol/dm³, and in water-stressed of the same specie ABA can reach 3.0 mcmol/dm³ (Wilkinson and Davis 1997).

ABA concentrations in animals as pigs ranged between 13-180 ng/100 g of fresh weight tissue in different organs. In rats fed with diet containing ABA, concentration was determined between 248-429 ng/100 g of fresh weight of brain tissue (Le Page-Degivry 1986, p. 1156). ABA concentration in animals is lower than it is found in plants.

ABA concentrations used in Dr. Livingston's experiments in vivo are higher than ABA concentrations usually found in plants. The most efficient group, showed in the experiment, was the number II, with 90% of survivors and a lower dose (1 mg/kg). Such concentration can be taken as a reference and defined as equivalent to 1 mg/ml. Nevertheless, doses can range between 0.1 mg/kg and 20 mg/kg, and concentrations between 0.1 mg/ml and 50 mg/ml. Doses may vary according to an application system.

ABA effect must be reproduced once again by repetitive doses, because ABA is metabolized and it induces a transient effect. ABA is involved in homeostatic mechanisms in plants, by which repetitive doses can produce a more prolonged effect.

### Preparation of a Buffer Solution

An ABA medication can be buffered at pH ranging between 5.2 and 6.5 for inducing cell normalization (86), and also can be buffered at pH ranging between 6.5 and 7.4 for inducing cellular apoptosis (88). An optimum pH 6.1 is elected to accomplish cell normalization and an optimum pH 7.0 is elected to accomplish cellular apoptosis. A pH for the buffer medication becomes equal to the pH of the bulk, because the buffer solution containing ABA will hold a pH of the extracellular medium until ABA is absorbed or as long as buffer capacity is permissible. Cancer progression decreases the normal blood pH (84), thus, application of an ABA buffer medication for inducing cell normalization would have a synergetic action in an acidic blood pH. ABA buffer medication for inducing apoptosis would be mostly recommended in patients with normal pH.

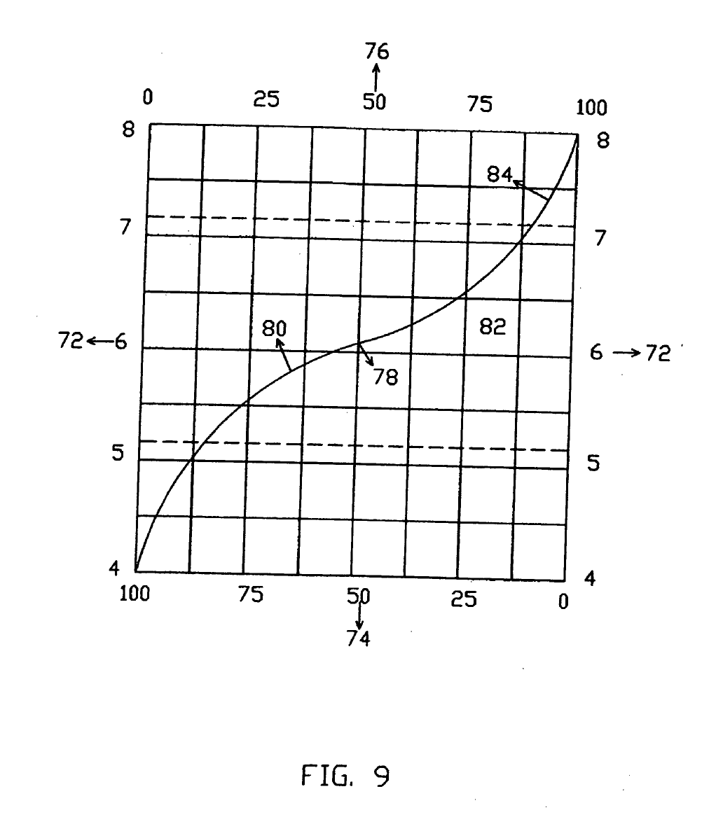

A buffer solution for the medication is initially prepared by using the method of Cassiday 1999, pp. 10-11. For the medication, it has been elected the pair carbonic acid and its salt (H₂CO₃—HCO₃⁻), which has a pK=6.1 (78). Graphic of the curve (80) has been showed in FIG. 9 as previous art. Curve is elaborated by the author by applying the Henderson-Hasselbalch equation and plotting values of pH between 4 and 7 (72), and, relative concentrations of carbonic acid (74) and bicarbonate (76) in percents. By far the most important buffer for maintaining acid-base balance in the blood is the carbonic acid-bicarbonate buffer. A simultaneous equilibrium reaction is given below:

H⁺+HCO₃⁻⇄H₂CO3⇄H₂O+CO₂

Buffer solution at pH 6.1 is fabricated by using 50% of carbonic acid and 50% of its salt. As pH=pK, buffer solution will have an optimal capacity inside of the region of maximum buffering capacity (82). Buffer solution at pH 7.0 is fabricated by using 12.5% of carbonic acid and 87.5% of its salt.

For the fabrication of an ABA buffer solution, it is important to get in count, the ABA dissociation and pH of the medication. Several researchers have mentioned that ABA is a weak acid that at neutral pH it occurs in a dissociated state; but at acidic pH, it occurs in the protonated form. ABA is highly dependent of the pH and apparently it dissociates as a strong acid. In an aqueous acid-base solution ABA equilibrium would be maintained by the carboxyl group and the carboxylate anion as follows:

ABA-COOH+H₂O⇄ABA-COO⁻+H₃O⁺

Due to that the carboxylate anion contained in ABA is more stable than the ABA carboxyl group the equilibrium favors the right side of the equation. Water as substitutive, near the carboxyl group acts to increase the acidity. It makes to ABA a stronger acid with bigger power of dissociation.

Research of Kelen et al. 2004, denominated “Separation of Abscisic acid, Indole-3-acetic, Gibberellic Acid in 99r (Vitis berlandieri×Vitis rupestris) and Rose Oil (Rosa damascena mill.) by Reversed Phase Liquid Chromatography” may help to determine and verify ABA dissociation according the pH. In this study it is considered that, retention in the mobile phase of carboxylic groups which are contained in these hormones depends on the percentage of ionized and non-ionized species. Results proved that, ABA is present in non-ionized form at approximately pH 4.0. Measurement of ABA pK value in the assay was calculated (pK=5.82) at 30% of acetonitrile. The ABA dissociation in the assay can be verified by using the Henderson-Hasselbalch equation as follows:

pH=pk+log [BASE/ACID]→4=5.82+log [BASE/ACID]

−1.82=log [BASE/ACID]→antilog(−1.82)=[BASE/ACID]

The result of the calculation is a proportion as follows:

[BASE/ACID]=0.015=0.15/100 [0.15% (BASE)−99.9% (ACID)*]

Also retention factors can be transformed in percent of non-ionized and ionized forms as follows:

| RETENTION | | % OF NON- | % OF IONIZED |
|---|---|---|---|
| FACTOR | pH | IONIZED (ABAH) | (ABA⁻ + H⁺) |
| 3.53 | 4 | 99.9* | 0.1 |
| 3.52 | 4.5 | 99.6 | 0.4 |
| 3.41 | 5 | 96.5 (100) | 3.5 (0) |
| 2.55 | 5.5 | 72.1 (70) | 27.8 (30) |
| 1.54 | 6.0 | 43.5 (40) | 56.4 (60) |
| 0.48 | 7.0 | 13.5 (10) | 86.4 (90) |

Above results were rounded to obtain a better view and correlation made below with research of Honberg et al. 1984.

Hornberg et al. 1984, pp. 321-323, found occurrence of a high-affinity guard cell specific ABA-binding proteins facing the apoplasmic space. They detected three different designated sites: as anion (one site) and for ABA in its protonated form (two sites). Such proportion means that, 30% of the receptor sites facing outside attach to ABA in its anionic form and 60% attach to ABA in its protonated form. Herein, it is strongly believed that a proportion of ABA receptor designated sites inside of cell is inverse or opposite to the Honberg structure of receptor designated sites facing the apoplasmic space.

Theoretically, it may be considered the receptor anionic site as an “ABA activator receptor site” which induces stomata closure and stomata opening. Likewise, the receptor protonated site may be considered as an “ABA carrier receptor site” which induces ABA absorption and transportation to the cell inside.

At pH 7, 90% of ABA dissociates, but only 30% is attached to the plasma membrane anionic site. It keeps an ABA plant economy in relation to the sequestration of the molecule. Most importantly, this mechanism stimulates: plant stomata closure when ABA attaches to outside anionic receptors, and, stomata opening when it attaches to same receptors into the cell inside. In transformed retention factors of Kelen et al. 2004, it can also be observed that, at pH 6, ABA attaches to both types of receptors inducing stomata closure and stimulating the molecule to be absorbed. Once, ABA protonated form gets across the plasma membrane, it becomes in its anionic form according to the ABA ion trapping concept. Thus, ABA⁻ will attach to anionic receptor sites inside of cell producing, a partial or a total phenomenon of stomata opening.

30% of receptor designated sites must be attached to ABA to produce the partial phenomenon and 60% to produce the total phenomenon.

For making ABA buffer solutions, it will be used an ABA dissociation percentage of 90% at pH 7, and, 60% at pH 6.1. For buffer solutions the ABA concentrations will range between 1 mg/ml and 3 mg/ml to produce a medication with a general concentration not higher than 0.9% W/V (9 mg/ml). The active principle (ABA) is soluble in methanol and is diluted at 50 mg/ml. Additional quantity of methanol, ethanol or another alcohol may be added to the medication to fulfill the pharmaceutical requirements. Molecular weights of the components are: ABA (264.3 gr), NaHCO₃ (84.01 gr), H₂CO₃ (62 gr). By adding ABA to the buffer solutions it will increase the acidity, which must be neutralized by the salt (sodium bicarbonate) according to the following reaction:

ABAH+NaHCO₃⇄ABA⁻+H₂CO₃+Na⁺

Because the reaction is equimolar, 1 mol of ABAH will react with 1 mol of NaHCO₃. The medication may be fabricated in different volumes: 5 ml injections, and 25 ml infuses, buffered at pH 6.1 and 7 (6.62) as follows:

| MEDICATION | ABA (mg/ml) | VOLUME (ml) | pH |
|---|---|---|---|
| 1 | 3 | 5 | 6.1 |
| 2 | 3 | 25 | 6.1 |
| 3 | 1 | 5 | 7.0 (6.62) |
| 4 | 1 | 25 | 7.0 (6.62) |

### Tween 80

In addition, the medication must include a quantity of between 1-10% of Polyoxyethylene Sorbitan Monooleate (CAS #9005-65-6) named also as TWEEN 80 (liquid formulation) and usually used as an emulsifier, solubilizer, surfactant, stabilizer, and dispersant. A preferred amount must be around a 10%, as maximum. In a solid formulation TWEEN 80 will be incorporated as powder. Also it can be used for manufacturing the medication similar products such as TWEEN 80 solution (10% low peroxide), TWEEN 80 solution 40×, Polysorbate 80, TWEEN 20 (Sorbitan Monooleate), and Polythylene Glycol Sorbitan Monooleate. TWEEN 80 is composed by Oleic acid, 60-70% (balance primarily linoleic, palmitic, and stearic acids). Those fatty acids show antioxidant properties. Oleic acid is an omega-nine fatty acid that, it is found naturally in many vegetable sources and animal products. It is considered as a healthy source of fat and is used as a replacement for high saturated animal fats. The mayor component in olive oil is the triglyceride ester of oleic acid. The research of Ismail M et al. 2010 titled, “Fatty acid composition and antioxidant activity of oils from two cultivars of Cantaloupe extracted by supercritical fluid extraction”, reveals the antioxidant capacity of linoleic and oleic acids.

The ABA molecule during the storage as pharmaceutical product is able to produce changes by oxidation and also by isomerization by ultraviolet light (UV) absorbance. The invention of Wang Number US 2008/0254988 A1, “Stable S-(+)-Abscisic acid liquid and soluble granule formulations”, indicates that by using brown PE bottles can be stopped the isomerization of ABA by UV light. In ABA experiments by using brown PE bottles, but not using any type of antioxidant or UV absorbent, the ABA degradation is only 8% in 33 days at 53 degrees centigrade. At ambient temperature and 21 months treatment produces a degradation of 75%. In the invention it is expressed that, by adding any type of antioxidant the degradation is zero. Above it is mentioned that TWEEN 80 contains oleic and linoleic acids which are able to block the oxidation products. Consideration of TWEEN 80 to be included in the medication, does not exclude an use of an antioxidant such as: lipoic acid, tocopherol or beta-carotene, which any of them can be included thereof.

### Medication Buffered at pH 6.1:

MEDICATION No 1: 3 mg/ml-5 ml H₂O-15 mg ABA/0.2 ml H₂O+0.3 ml methanol (113.5 mM-dissociated at [60%] 68.1 mM)-15 mg sodium bicarbonate/4.5 ml H₂O (39.6 mM)+15 mg carbonic acid/4.5 ml H₂O (53.7 mM). After adding ABA the medication molarity is 28.3 mM (base) and 55 mM (acid) calculated it for 5 ml. The pH of the initial medication is verified and calculated through the Henderson-Hasselbalch equation as follows:

pH=pka+log [BASE/ACID]=6.1+log [28.3 mM/55 mM]=5.81.

As the resultant pH is under the expected value (6.1), the pH of the medication is readjusted at pH 6.1. New concentrations of the base and acid are as follows: 20 mg sodium bicarbonate (52.9 mM)+10 mg carbonic acid (35.8 mM). Final molarity is 40.8 mM (Base) And 39.0 mM (acid). The pH is recalculated:

pH=6.1+log [40.8 mM/39.0 mM]=6.1

MEDICATION No 2: 3 mg/ml-25 ml H₂O-75 mg ABA/1 ml H₂O+1.5 ml methanol-99.0 mg sodium bicarbonate/25 ml H₂O+51 mg carbonic acid/25 ml H₂O, Same concentrations, final molarity and pH as in medication No 1.

### Medication Buffered at pH 7 (6.62):

MEDICATION No 3: 1 mg/ml-5 ml H₂O-5 mg ABA/0.4 ml H₂O+0.1 ml methanol (37.8 mM-dissociated at [90%]34.0 mM)-35 mg sodium bicarbonate/4.5 ml H₂O (92.5 mM)+5 mg carbonic acid/4.5 ml H₂O (17.9 mM). After adding ABA the medication molarity is 76.4 mM (base) and 22.8 mM (acid) calculated for 5 ml. The pH of the medication is verified as follows:

pH=pka+log [base/acid]=6.1+log [76.4 mM/22.8 mM]=6.62

This resultant pH is not recalculated because, by increasing the percent of the base and decreasing the percent of the acid, the pH of the medication would be out of the region of maximum buffering capacity (see FIG. 9). Thus, it decreases the efficiency of the medication, however, the obtained medication pH is close to the expected value (7.0) and it is found inside of the range for inducing cellular apoptosis (6.5-7.4).

MEDICATION No 4: 1 mg/ml-25 ml H₂O-25 mg ABA/2.0 ml H₂O+0.5 ml methanol-175 mg sodium bicarbonate/25 ml H₂O+25 mg carbonic acid/25 ml H₂O. Same concentrations, final molarity and pH as in Medication No 6.

### Embodiment Number One

A pharmaceutical medicine for an intravenous, intramuscular and subcutaneous or intraperitoneal treatment may be elaborated by using the buffer system as mentioned before. ABA concentrations can range between 0.1 mg/ml and 5 mg/ml and best range would be between 1 mg/ml and 3 mg/ml. Medication will be prepared by obtaining an isotonic solution not higher than 9 mg/ml (0.9% W/V). Doses range between 0.1 mg/kg and 5 mg/kg. Liquid volume of the medications may vary between 5 ml (injections) and 5 ml (infuses). ABA in plants is conducted from roots to leaves via across the xylem, by which the ABA best application for a cancer treatment would be by intravenous way. ABA application via the human circulatory system would mimic ABA hormonal flow in plant streams and xylems.

### General Liquid Composition

An ABA pharmaceutical liquid composition in general terms, could be conformed by the following substances:
Active principle
Buffer ingredients (Acid and salt of the acid)
Solvent of the active principle
Solubilizer, emulsifier, surfactant, dispersant and antioxidant
Diluent of the solution

ABA Pharmaceutical Liquid Compositions of Embodiment Number One

Pharmaceutical liquid compositions of the medication No 1 and No 2 are as follows:
Medication No 1
ABA concentration=3 mg/ml.
Volume=5 ml
ABA=15 mg (33.3% W/W)-113.5 mM. Dissociation 60% (68.1 mM)
Sodium Bicarbonate=20 mg (44.4% W/W)-40.8 mM.
Carbonic Acid=10 mg (22.2% W/W)-39.0 mM.
Methanol=0.3 ml (6% V/V)
Distilled Water=4.2 ml (84% V/V)
TW80=0.5 ml (10% V/V)
pH=6.1
Medication No 2
ABA Concentration=3 mg/ml.
Volume=25 ml
ABA=75 mg (33.3% W/W)-113.5 mM. Dissociation 60% (68.1 mM)
Sodium Bicarbonate=99.0 mg (44.4% W/W)-40.8 mM.
Carbonic Acid=51 mg (22.2% W/W)-39.0 mM.
Methanol=1.5 ml (6% V/V)
Distilled Water=21 ml (84% V/V)
TW80=2.5 ml (10% V/V)
pH=6.1

Pharmaceutical liquid compositions of the Medication No 3 and No 4 are as follows:
Medication No 3
ABA Concentration=1 mg/ml.
Volume=5 ml
ABA=5 mg (11.1% W/W)-37.8 mM. Dissociation 90% (34.0 mM)
Bicarbonate=35 mg (77.7% W/W)-76.4 mM.
Carbonic Acid=5 mg (11.1% W/W)-22.8 mM.
Methanol=0.1 ml (2% V/V)
Distilled Water=4.4 ml (88% V/V)
TW80=0.5 ml (10% V/V)
pH=6.62
Medication No 4
ABA Concentration=1 mg/ml.
Volume=25 ml
ABA=25 mg (11.1% W/W)-37.8 mM. Dissociation 90% (34.0 mM)
Bicarbonate=174.8 mg (77.7% W/W)-76.4 mM.
Carbonic Acid=24.9 mg (11.1% W/W)-22.8 mM.
Methanol=0.5 ml (2% V/V)
Distilled Water=22 ml (88% V/V)
TW80=2.5 ml (10% V/V)
pH=6.62

An ABA pharmaceutical liquid composition, not buffered and in general terms, could further include a combination of the following substances:
Active principle
Solvent of the active principle
Solubilizer, emulsifier, surfactant, dispersant and antioxidant
Diluent of the solution

Pharmaceutical liquid compositions of the medication No 5 (not buffered):
Medication No 5
ABA Concentration between 1 mg/ml and 3 mg/ml.
Volume between 5 ml and 25 ml
ABA between 5 mg and 75 mg
Methanol between 0.1 ml and 1.5 ml
Distilled Water between 4.2 ml and 22 ml
TW80 between 0.5 ml and 2.5 ml

Liquid formulations included in the present invention can be also fabricated in the forms of an emulsion, and an aqueous or oily suspensions. Also it is not excluded transdermal patches medications, or oral medications which it is prepared as a liquid composition, or in the form of syrups or elixirs, and any other form suitable for use.

### ABA Decay Length in Xylem

Efficiency of an ABA intravenous treatment must incorporate variables such as speed of flow, permeability and loses by absorption via the circulatory system. ABA decay length in xylem (Lxylem) has been studied by Kramer 2006. Specifically in xylem SAP this factor will depend of the ABA traveling speed in xylem (V), radius of the xylem vessel (R), membrane permeability of ABAH (P_{AH}), and ABA effective permeability of sink cells (Peff). Kramer equations are defined as follows:

(Lxylem)=1.15RV/Peff WHERE Peff=P_{AH}(1/1+10^{pH-pKa})

According to Kramer 2006, page 1235, in the absence of carriers, most of the weak acids have a L-xylem of approximately 2 m or greater. Also in this research, it is pointed out that ABA L-xylem reaches 10 m with variations between 2.2 m at 1 m/h speed and 22 m at 10 m/h speed. L-xylem is proportional to the speed of flow in the xylem. That means that, ABA losses in xylem are proportionately higher at low transpiration rates. If it is introduced a catheter in the arm until it reaches the heart, the pass over distance is about 50 cms. The ABA protonated form in xylem, can reach a distance in average 20 times higher than that distance before it becomes absorbed, trapped or metabolized. Kramer equations do not take in count a traverse distance in xylem of the ABA dissociated form. ABA can be considered a hormone that, might send a long range signaling which, is compatible with its function and role against cancer and stress.

### Embodiment Number Two

Le Page-Degivry et al. 1986, demonstrated in the article “Presence of Abscisic Acid, A Phytohormone in the Mammalian Brain”, that ABA as molecule keeps its structure and properties after it is consumed. In such experiment, rats were fed with an ABA containing diet. Tissue ABA determination by radioimmunoassay, after experiment, detected hormone concentrations between 248 to 429 ng/100 g of fresh weight tissue.

Dr. Livingston's experiment using mice also proved that, an use of ABA by “oral via” had efficiency killing myeloid leukemia. Nevertheless, better results during the Dr. Livingston's experiment were obtained with a dose at 100 mg/kg (90% of survival) than a dose at 10 mg/kg (60% of survival).

Concentrations used to manufacture capsules can range between 1 mg/gr and 100 mg/gr and 10 mg/kg and 100 mg/kg bodyweight.

ABA active principle can be prepared by using an acceptable carrier as a vehicle, and packing such principle in capsules with a biodegradable dark coating to avoid isomeric changes due to the light (both as mentioned below).

### ABA Pharmaceutical Solid Compositions of Embodiment Number Two

An ABA medical composition also includes a solid composition fabricated in capsules of 250 mg (total weight) at 40 mg/gr ABA concentration, for inducing cell normalization and apoptosis. In it has been included a pharmaceutical carrier as microcrystalline cellulose I.P., which serves as a pharmaceutical carrier (thickening and vehicle agent):

For Inducing Cell Normalization:
ABA 10 mg at (4% W/W)
Sodium Bicarbonate 50.0 mg at (20% W/W)
Carbonic Acid 50.0 mg at (20% W/W)
Methanol 0.2 mg at (0.08%) as primary solvent of ABA prior a preparation
TW80 in powder formulation 2.5 mg at (1%)
Distilled water 2.5 mg at (1%) in capillary form of water
Microcrystalline Cellulose I.P. as pharmaceutical carrier 134.8 mg at (53.9% W/W)

For Inducing Cellular Apoptosis:
ABA 10 mg at (4% W/W)
Sodium Bicarbonate 87.5 mg at (35% W/W)
Carbonic Acid 12.5 mg at (5% W/W)
Methanol 0.2 mg at (0.08%) as primary solvent of ABA prior a preparation
TW80 in powder formulation 2.5 mg at (1%)
Distilled water 2.5 mg at (1%) in capillary form of water
Microcrystalline Cellulose I.P. as pharmaceutical carrier 134.8 mg at (53.9% W/W)

The active ingredients of the solid pharmaceutical composition can include a combination of the mentioned ingredients, for the fabrication of essentially dark coating capsules, which it requires to block the UV light for avoiding the isomeric changes of the ABA molecule. This formulation does not exclude the manufacturing of the medication based in tablets, pellets, suppositories and caplets. Also it is not excluded oral solid medications, such as in powder or where it is prepared in any other form suitable for use.

### Microcrystalline Cellulose I.P. as Pharmaceutical Carrier

Microcrystalline cellulose (MC) is a natural occurring substance that, it has been proven to be stable, safe and physiologically inert. It is fabricated with purified and partially depolymerized alpha cellulose. MC has the capacity of showing compressibility and carrying capacity. It exhibits excellent properties, as an excipient for solid dosage forms. It compacts well under minimum compression pressures. Other advantages include low friability, inherent lubricity and good dilution potential. MC from Sancel Company could be valuable as filler and binder for formulations prepared by direct compression to make tablets or for pelletisation. MC is derived from a special grade of alpha cellulose. MC is comprised of glucose units connected by a 1-4 beta glycosidic bond. The linear cellulose chains are bundled together as micro fibril spiraled together in the walls of plant cell. Each micro fibril exhibits a high degree of three-dimensional internal bonding resulting in a crystalline structure that is insoluble in water and resistant to reagents. There are, however, relatively weak segments of the micro fibril with weaker internal bonding. These are called amorphous regions but are accurately called dislocations since micro fibril containing single phase structure.

MC is produced at a size of 50 micron (Sancel 101). This material is white, odorless, tasteless, and free from organic and inorganic contaminations. It is also insoluble in water and sodium hydroxide, dilute acids, and in mostly all organic solvents.

MC also is highly absorptive due to the capillary action of its surface porosity, making it possible to act as a carrier for liquids and yet retain free flowing and compression properties. Its porosity promotes easy wetting and rapid drying of wet granulation.

MC has no specific requirement for storage. It may absorb moisture if exposed to the atmosphere with a relative humidity higher than 65%. MC is packed in net 25 Kg in HDPE woven sack bag with inside two HM liners.

MC is primarily designed for direct compression tablet making, nevertheless, it can be adapted for making gelatin capsules to receive accurate dosed of dry powder medication. The dry powder may be formed into coherent masses of approximately capsule size by blending and pressing with the aggregates, thus avoiding the necessity of handling dry powder in connection with the filing and cleaning of the capsules. The pressed capsule charge may then be placed as one piece in the capsules and may provide a more positive flow through the hoppers of the capsule filling equipment.

Some MC pharmaceutical specifications are: Appearance (fine or granular, white or almost white powder; pH 5-7.5; Starch and dextrins (absent); Organic impurities (comply); Organic volatile impurities (not applicable); Water Soluble Substances % NMT (0.2). MC concentration would be at 53.9% W/W.

### Others Pharmaceutical Carriers for an ABA Solid Composition

An ABA pharmaceutical solid composition also may include Sodium Starch Glycolate (SSG), which is the sodium salt of a carboxymethyl ether of starch. The appearance is very fine, white or off white, free flowing powder, odorless or almost odorless. It is practically insoluble in water and in most organic solvents. It consists of oval or spherical granules, 30-100 micron in diameter with some less spherical granules ranging from 10-35 micron in diameter. SSG is widely used in oral pharmaceuticals as a disintegrated in capsule. The recommended formulation is 2-8%, with the optimum concentration about 4% although in many cases 2% is sufficient. This material can be used for an ABA solid medication in such case that, a buffer composition is not applied.

Other material that can be included as pharmaceutical carrier in an ABA solid medication is Talc (Mg-Silicate) with residue of magnesite, dolomite and chlorite. The pharmaceutics specifications of the Microtalc Pharma 30 from Mondo Minerals B.V manufacturer are: 96% Mg-Silcate; CAS-No. 14807-96-6; Water soluble <0.2%; pH 9; Brightness Ry 93%; Refractive Index 1.57; Median particle size 8 micron; Packed Bulk density 0.65 g/cc; Moisture 0.2%.

In addition, others pharmaceutical acceptable carriers can include glucose, lactose, gum acacia, gelatin, mannitol, urea, dextrins. Concentration of above carriers in capsules would be at 53.9% W/W.

### Dark Enteric Coating of Hard Gelatin Capsules

Dark coated capsules can be a suitable form for protecting the ABA molecule against isomeric changes of the red and UV light, which can occur thereof.

Capsule can be fabricated with enteric coatings. They are in particular used to: protect active substances destroyed by the acidic gastric juice; improve tolerability of medicaments, which can irritate the stomach by only releasing them in the small intestine; help to achieve targeted release and concentration in the small intestine. Enteric coating can be also used to stabilize acid-sensitive medicaments (Thoma K et al. 2000).

Formulations for enteric film coating contain the following main components: enteric film formers; plasticizers; anti-adhesion agents, colorants or pigments; solubilizers or dispersion agents and other additives. The film formers mainly in use today are polymers with carboxyl groups, which are water insoluble in the protonated state, and pass into solution in the weakly acid to neutral range, between pH 5 and 6.5 through formation of salts (Thoma K et al. 2000).

Film formers that can be used for the manufacturing of hard gelatin capsules are essentially made of: polymethacrylates; cellulose-based polymers; polyvinyl derivatives and other copolymers made of half esters.

The pH solubility of the coating film, which determines its dispersion and absorption in the body, is an important factor for the election of the hard gelatin capsules. Coating film formulation that, can be recommended for the ABA solid composition in capsules for inducing cell normalization at pH 6.1 and cellular apoptosis at pH>6.62 are based in cellulose derivatives. The Cellulose Acetate Phthalate (CAP) film formulation, produces a solubility of the medication at a pH range in between 6.2 and 6.5, and may be recommended for inducing cell normalization. The ingredients of the CAP formulation are: CAP 5.56% by weight; diethyl phthalate 3.34%; isopropanol 22.78%; and methyl chloride 68.32%. The Hydroxypropylmethylcellulose acetate succinate (HPMCAS) film formulation, specifically the option HF, produces solubility at pH>7, and may be recommended for inducing cellular apoptosis of the medication.

For film coating pigmentation is used: titanium dioxide, pigments based in foodstuff lakes and iron oxide. In the case of the ABA hard gelatin capsules, it must be elected a dark or opaque film to avoid isomeric changes. Instruction recommendations use the smallest possible pigment particles sizes (<15 micron), to obtain smooth films. Capsule film coating machine, as the BY400 from Lonkou Leading Machinery Co Ltd, can be used for the performance of the pharmaceutical operations.

### Embodiment Number Three

ABA catheter induction to an inoperable tumor location, for inducing senescence, can be considered an alternative method to control terminal types of cancer. Nevertheless, it may result a more expensive treatment. In causing senescence against cancer, the treatment brings itself an use of an ABA massive dose. Hormone concentration in the medication may be diluted in methanol or ethanol at 50 mg/ml, and administered with or without buffer by using a catheter. It will permit a fluid passage of the hormone to be directly applied to a tumor.

A (+/−)-Abscisic acid manufactured by Sigma-Aldrich is able to carry the mentioned concentration in ethanol which may be clear to slightly hazy. Such synthetic form of ABA (CAS Number 14375-45-2) is obtained through a plant cell culture tested with 99% of purity. A liquid combination for catheter induction may be elaborated at ABA concentrations in methanol in between 10 mg/ml and 50 mg/ml. The medication includes the following components and ranges:
Between 10 mg and 50 mg of ABA
Between 0.1 ml and 1.5 ml of methanol
Between 35 mg and 99 mg of sodium bicarbonate
Between 5 mg and 51 mg of carbonic acid
Between 0.5 ml and 2.5 ml of TWEEN 80
Between 5 ml and 20 ml of distilled water

### Embodiment Number Four

When parenteral nutrition (PN) is necessary and required in debilitated cancer patients, it may be recommended an ABA administration of the plant hormone using a vehicle as dextrose/glucose in saline solution. Although it has been largely debated, PN is able to feed a person via intravenously, bypassing the usual process of eating and digestion. The person receives nutritional formulae that, contains nutrients such as glucose, amino acids, lipids, vitamins and dietary minerals. By adding ABA, the medication will be useful for nutrition as well as treating the disease of cancer, especially for patients where the organism and immune system has been weakened, for example, by using chemotherapy and radiation. The medical composition can be prepared by using PN as a pharmaceutical liquid carrier.

The types of PN which can be used are: (D5W) 5% dextrose in water or (G5W) 5% glucose in water; (DSNS) 5% dextrose in normal saline 0.90% WN of NaCl or (G5NS) 5% glucose in normal saline 0.90% W/V of NaCl; (D51/2NS) 5% dextrose in half amount of normal saline 0.45% W/V of NaCl or (G51/2NS) 5% glucose in half amount of normal saline 0.45% W/V of NaCl. The containing of the medication must be tored in brown bottles. The medical composition would contain the same ingredients as the main combination, but adding the dextrose/glucose saline physiological solution as pharmaceutical carrier, which it contains a concentration of dextrose or glucose at 50 mg/ml of water to give 5% dextrose or glucose concentration. The pharmaceutical product would have a volume in between 51 and 76 ml. A preferred medication would contain in between 50 mg and 75 mg of ABA, to give 1 mg/ml and 1 mg/kg of bodyweight of ABA:
between 5.0 and 75.0 mg abscisic acid
between 20.0 and 174.8 mg sodium bicarbonate,
between 5.0 and 51.0 mg carbonic acid,
between 0.10 and 1.5 ml methanol,
between 4.2 and 22.0 ml distilled water
between 0.5 and 2.5 ml TW 80,
between 25 and 50 ml of the saline physiological solution as pharmaceutical carrier

### Embodiment Number Five

ABA also may be delivered as an oral liquid composition, contained and packaged in liquid filled soft gel capsules, with a dark enteric coating of hard gelatin to avoid isomeric changes by light. The coating will produce a liquid release of ABA in the small intestine. Capsules can contain a volume of 1 ml or more, thus, ABA concentration in the main medication must be increased in between 76 mg and 650 mg of ABA. Concentration of the plant hormone will be ranging in between 2.9 mg/ml and 25 mg/ml. A preferred medication would contain 650 mg of ABA in 26 ml volume of distilled water. The formulation is able to produce 26 capsules of 1 ml each (25 mg per capsule and 0.5 mg/kg of bodyweight for a patient with 50 kgs). The rest of the ingredients will remain with same concentration as in the main composition as follows:
between 76.0 and 650.0 mg abscisic acid
between 20.0 and 174.8 mg sodium bicarbonate,
between 5.0 and 51.0 mg carbonic acid,
between 0.10 and 1.5 ml methanol,
between 4.2 and 22.0 ml distilled water
between 0.5 and 2.5 ml TW 80

### ABA Production, Cost of Medication and Handling

Production of ABA by using the fungus Cercospora rosicola as mentioned by Assante et al. 1977, pp. 1556-1557, can carry out a commercial level. A strain denominated, Cercospora rosicola Passerini, frequently found on Rosa sp, produces 6 mg/100 ml maximum of ABA. It must be cultivated on a potato-agar medium, at pH 6.5-6.8, under 24 degrees Centigrade in the light for 30-40 days.

ABA production obtained from C. rosicola is considered high, whether it is compared with ABA yields from plant materials. Addicott et al. 1969, p. 142, showed that such yields of plants ranged between 7 and 40 mcg/kg. A higher yield of 9 mg was obtained by processing 225 kg of dry weight from Gossypium fruit, which yielded 40 mcg/kg.

Other strains of ABA fungi sources include Cercospora cruenta, Botrytis cinerea, Ceratocystes coerulescens, C. fimbriata, Fusarium oxysporum, and Rhizoctonia solani (Zeevaart et al. 1988). An invention of the Chengdu Institute of Biology Academy of Sciences named, “A New Process for Preparing Natural Abscisic Acid” 2008, uses a fungus to isolate ABA, through a method of fermentation.

From Sigma-Aldrich, different forms of ABA (CAS Number 21293-29-8, molecular weight 264.32) can be purchased, such as: (+) Abscisic acid 99% purity as the natural occurring; (+/−) CIS, trans-Abscisic acid-³H (G) 95% purity as synthetic substance; (+/−) Abscisic acid 98-99% purity as synthetic substance; (+) Abscisic acid 98% purity as natural isomer; (−) CIS, trans-Abscisic acid as racemic or enantiomer. ABA price listed for controlled laboratory use oscillates around $60/100 mg. The cost of each ABA buffered medication is as follows:

| MEDICATION | ABA (mg/ml) | VOLUME (ml) | ABA (mg) | COST ($) |
|---|---|---|---|---|
| 1 | 3 | 5 | 15 | 9 |
| 2 | 3 | 25 | 75 | 45 |
| 3 | 1 | 5 | 5 | 3 |
| 4 | 1 | 25 | 25 | 15 |
| CAPSULES | 40 mg/gr | 250 mg (W) | 10 | 6 |

Pharmaceutical material must be handled under protection from light and at specified and variant storage temperature depending of the ABA used material.

### Side Effects

ABA definitively cannot be prescribed during pregnancy due to existence of hCG in placenta. ABA, being able of neutralizing hCG might cause abortion and also may inhibit and interfere in mammal reproduction. An efficacious medicine against hCG and cancer must have a similar effect against human reproduction, due to the reported presence of hCG in placenta, human fetus, embryos and human spermatozoa. According to H. Acevedo 2002, malignant transformation and human reproduction share common genetic (evolutionary) and biochemical pathways related to hCG. Moreover, ABA has been found and related to inhibition of insect reproduction in low dosage amounts, and having a direct ovicidal effect (U.S. Pat. No. 4,434,180 of Visscher S. N 1984). Also in agreement to Verhaert P and DeLoof A 1986, a peptide similar to the vertebrate gonadotropin has been described in the central nervous system of the cockroach (Periplaneta americana). Correlation of former statements produces the strong indication that, ABA could harmless interfere in human reproduction under a natural and variant way in direct relation to the ABA fluctuating levels in human blood. The following researches have determined that, whether it is not considered the above mentioned side effects, ABA has no any side effects:

1—Dr. Livingston reported during experimental observation, that ABA apparently had no toxic side effects in mice, even when administered (i.p) in amounts up to 10% by weight of mice. Thus, considering 28 g body weight mouse and ABA (i.p) administered at 2800 mg per week, ABA had no adverse side effects.

2—Hong et al. 2006, of the Chengdu Biological Institute Academy of Sciences, mentions in a direct and undoubtly form, in the abstract of the invention number (CN 1748674A) that, ABA had just a very little effect in animals and treated cells.

3—Herrero M. P and Shafer W 2012, from Valent Biosciences also reveal that, ABA had no side effects during the experiments. This conclusion was determinated in different animal experiments and in three different U.S. inventions.

4—Liang F. S et al. 2011, from the Howard Hughes Medical Institute of the Stanford University, in the research titled “Engineering the ABA plant stress pathway for regulation of induced proximity”, expressed that, ABA may be well suited for therapeutic applications and as experimental tool to control diverse cellular activities in vivo. ABA has an acute oral median lethal dose (LD₅₀) of >5000 mg/kg in rat and a “no observable adverse effect level” (NOAEL) of 20,000 mg/kg per day in sub chronic toxicity studies reported by the EPA.

### Active Principle Election (Natural Vs Synthetic)

During the fabrication of the medication, the election of the ABA active ingredients is important. It has been pointed out, differences in ABA catabolism and uptake, between natural ABA and racemic compounds. In accordance to Cutler A. J et al. 1999, ABA has been reported to be in plants a unstable compound due to active enzymatic degradation. According to Mertens R et al. 1982, in leaf discs of V. Faba, natural ABA (S) was catabolized much more rapidly than the racemic ABA. The ABA half-lives were 6-8 hours and 30-32 hours, respectively (cited in Zeevaart JAN A. D et al. 1988). In order to examine the ABA stability in mammalian cells, Liang F. S et al. 2011, elaborated experiments with Chinese hamster ovary (CHO) cells for up to 48 hours. They assayed the functional concentration of the hormone by the ability of the ABA-cell culture medium to induce luciferase. After 24 hours of incubation with cells, about 60% of ABA activity was retained, and after 48 hours, the activity was reduced to around 23%. For potential applications in humans, they examined the stability of ABA in isolated serum by incubating ABA with fresh human serum or heat-inactivated fetal bovine serum for up to 48 hours. Liang F. S et al. 2011 found that, ABA was stable in serum for up to 48 hours, retaining about 64 to 77% activity. When ABA was injected intraperitoneal, it entered in the circulatory system rapidly, and activity was detectable within 30 min. When ABA was administered orally it had a half-life of about 4-hours. In accordance to the studies of Liang F. S et al. 2011, a medication must be fabricated considering a natural ABA form instead a synthetic form.

### ABA Metabolism in Relation to Cancer

ABA is used as cytokine against cancer cells in a healthy aerobic metabolism, and the principle is not produced in a sick anaerobic metabolism, because the existence of a maintained lack of oxygen in blood of cancer patients.

According to Dr. V. Livingston (U.S. Pat. No. 3,958,025, 1976), ABA presence has been demonstrated in human serum and urine by the R. F. Scand, Jr. Clinic Laboratory Investigation on January 1970. On the same paragraph, it says that, the serum of healthy persons can be demonstrated to have a higher inhibiting effect on plants than that of the sick or afflicted; that is, a greater amount of the inhibitory factor exists in the blood of the well person. In invention of Zocchi et al. 2008, page 3, presence of ABA in human plasma was determined by HPLC-coupled mass spectrometry (HPLC-MS). It was found ABA plasma concentrations in the range of 5-10 nM, of both, the cis-trans isomer (the active form in plants), and the trans-trans isomer (inactive form).

According also to V. Livingston, ABA as vitamin A derivative is produced by the liver. This reaction essentially might be demonstrated by adding carrot juice, which contains Beta-carotene, to liver powder. A healthy liver contains the required enzymes to produce appreciable amounts of ABA. Apparently, the ABA amounts consumed by digestive via are not consistent with the liver internal plant hormone production. It has been proven in healthy animals that, although a diet could be low in ABA, still the ABA internal production is high. According to Le Page-Degivry et al. 1986, animals fed on ABA poor diet had more ABA in their brains than did control animals.

ABA is not degraded in an organism deficient in oxygen. ABA 8-hydroxylase (CYP 450 enzyme) catalyzes the first step in the oxidative degradation of ABA. This mechanism, studied in plants by the National Research Council of Canada, requires oxygen. The ABA biosynthesis also requires oxygen. Oxygen is incorporated, in order to become the Abscisyl aldehyde molecule form (FIG. 21) to the ABA form (FIG. 1), through the abscisyl aldehyde oxidase enzyme. ABA biosynthesis and degradation, in plants and animals, are encompassed metabolic processes, in order to maintain an equilibrium. ABA has been correlated in plants with homeostatic mechanisms.

ABA discovery can be analyzed under the standpoint and theory of R. Schreiber's cancer immunoediting. Low concentrations of ABA and oxygen in blood of cancer patients, produce a cancer survival (evasion form); meanwhile adequate ABA concentrations and oxygen, in a healthy metabolism, allow the immune system to destroy cancer (elimination form). An equilibrium form, between immune cells and cancer cells, is produced through a complex adaptive mechanism interaction. ABA concentrations and biological activity, oxygen pressure in blood, and cancer immune resistance and mutation, may be important variables in a cancer interactive equilibrium system with immune cells.

### ABA As Inductor of a Change of Metabolism and Types of Cancer

In cancer, the tumors are the symptoms of the disease and not the real cause of the pathologic manifestation of the sickness. As said along this invention, the real cause of cancer is a change of metabolism from an aerobic toward an anaerobic glycolytic metabolism pointed out by O. Waburg. Although these changes occur, paradoxically the internal cancer metabolism, at cellular level, switches later on and during the last stages of the disease toward an aerobic condition. ABA as an inductor of a metabolic change, reverse a damage acquired metabolism producing a replacement of sodium by potassium. Enough research has been supplied in this invention about it, nevertheless, consequences in the incidence of cancer correlated by those physiological changes have not.

Metabolic movements of Na⁺ and K⁺, in relation to cancer etiology, have been studied by different researchers by indicating, a correlation between sodium increase in human tissue and a notable incidence of cancer (Jansson B et al. 1975, 1978, 1981, 1985, 1986, 1987, Pantellini G. V et al. 1976, Newmark H. L et al. 1985, Tatsuta M et al. 1991, Jacobs B 1990). In accordance to the mentioned researches, and as it could be observed along of this invention, ABA is able to counteract and reverse this phenomenon, denominated by Cope in 1978, the tissue damage syndrome. Papers of Pantellini G. V et al. 1976 claim that, replacement of potassium ions by sodium ions in nuclei acids loosens up the stability of DNA and RNA, stimulating the opening of valence bonds and causing the formation of neoplasms. Research of Jansson B et al. 1996 demonstrated that, in areas where a consumption of sodium is more elevated the cancer incidence is higher, meantime, in areas where the consumption of potassium is higher the cancer incidence is lower, for example, the Seneca County in the State of New York; the reasons are given in the study. The types of cancer submitted by the study were colorectal cancer, breast cancer, gastric cancer and all malignant neoplasms.

Specifically, ABA produced a severe effect against different cancer cell lines: HELA (cervical cancer); DU 145 (prostate cancer); HCT115 (colon cancer); K562 (leukemia), C1498 (myeloid leukemia), HL-60 (promyelocytic leukemia); SMMC-7721 (hepatocarcinoma); and Tca8113 (squamous oral cell carcinoma).

In addition to the types of cancer already mentioned, which can be prevented and controlled by ABA, there are several other types of cancer that, could be target subjects of an ABA treatment: adrenocortical carcinoma; AIDS-related cancers; skin cancer and melanoma; extrahepatic bile duct cancer; bladder cancer; bone cancer and chordoma; Ewing sarcoma; non-Hodgkin lymphoma; heart tumors; ductal carcinoma; endometrial cancer; esthesioneuroblastoma; extracraneal germ cell tumor; extragonadal germ cell tumor; retinoblastoma; gallbladder cancer; cancer soft tissue sarcoma; gestational trophoblastic tumor; head and neck cancer; Hodgkin lymphoma; throat cancer; pancreatic cancer; kidney cancer; Wilms tumor; laryngeal cancer; pheochromocytoma; parathyroid and thyroid cancer; penile, testicular, vaginal, vulvar, ovarian and uterine cancer; lobular carcinoma; lung cancer and pleuropulmonary blastomas; malignant mesothelioma; midline tract cancer; multiple endocrine neoplasia; pituitary tumor; transitional cell cancer; rhabdomyosarcoma; urethral cancer; thymoma; and benign tumors and other related diseases of abnormal mass growing tissues.

### Operation

Radiation or chemotherapy administered before, during or after ABA treatment is not recommended, because those traditional treatments weaken the patient's immune system. A simultaneous and coordinated action adopted from a patient and physician, strengthening the immune system and stimulating an attack of immune cells, will be an indicated treatment against cancer, under the specifications of this invention. Also, a patient diet with high consumption of carotenoids (the ABA precursor) is highly recommended.

Destruction of cancer tissues by any treatment brings also a process of released toxins, which might poison other tissues of the body. Therefore, it is recommended during a patient recovery process, a method for detoxifying the human organism during ABA treatment. It also is advisable to start an ABA treatment, after large tumors have been removed by surgical procedures. Physicians can elect two different ABA buffer medications, which they may produce a differential toxin unload to the blood patient. It is recommended an use of an ABA buffer medication for inducing cell normalization to: patients with a critical health condition; in terminal types of cancer; patient with inoperable large tumors; for elderly patients and for those patients in which toxins can compromise the patient life or vital organs; and for patients with damaged organs of excretion as colon, kidneys, and liver. Likewise, it is recommended ABA buffer medication for inducing apoptosis: in early stages of cancer; for young patients; and in those patients with small tumors, where toxin unload, released from cancer cell destruction or apoptosis, does not compromise the life of a cancer patient.

### CONCLUSIONS

Clearness about existing compatibility, between ABA as a medicine to fight cancer and the disease, is evident and can be perceived through the invention. Any medicine proposed for cancer must come into the conjunction of the recovery process mechanism of Dr. Gerson.

Ideas, theories and references of the invention can be used as tools for better understanding the nature, so as expressed in general terms by Ho in 1993: “As in any attempt to understand, we use whatever tools we have at our disposal to help us think, and good scientific theories are just that, a superior kind of tools for thought”.

It will be apparent to those skilled in the art that, modifications can be made without departing from the object and scope of the present invention. Therefore, it is intended that the invention only be limited by the claims.

What is claimed is:

1. A medical solid composition improved with a coating for treating mammals in a determined amount upon a damaged cell, using the coating to provide protection for the composition, as the coating being insoluble in the stomach and producing a targeted release concentration of the composition in the small intestine, comprising the medical solid component of the composition:
between 5.0 and 75.0 mg abscisic acid,
between 20.0 and 174.8 mg sodium bicarbonate,
between 5.0 and 51.0 mg carbonic acid,
between 0.10 and 1.5 ml methanol,
between 4.2 and 22.0 ml distilled water,
between 0.50 and 2.5 ml TW 80,
whereby said composition is coated in capsules using a titanium dioxide dark pigmented hard enteric gelatin made with a cellulose acetate phthalate polymer, and said coating comprising: a 5.56% cellulose acetate phthalate by weight, diethyl phthalate at 3.34%, isopropanol at 22.7%, and a methyl chloride at 68.32%,
whereby said dark pigmented coating of titanium dioxide avoids that the abscisic acid molecule becomes inactive by light isomerization by moving carbon 1 in cis configuration position of abscisic acid as shown in FIG. 10, to a trans configuration position of said carbon 1 as shown in FIG. 51,
whereby said coating is insoluble at an acidic pH 3 of the gastric environment, and the composition is kept encapsulated during a transit of the medication in the stomach,
whereby a pH in the small intestine varies in between 5 and 9,
whereby a cellulose acetate phthalate coating, which dissolves at a pH in between 6.2 and 6.5, stimulates a targeted release of a hydrogenated lipophilic form of abscisic acid in the small intestine for inducing cell normalization,
whereby a determined cellulose acetate polymer coating, which dissolves at a pH 7 and higher, stimulates a targeted release of a dehydrogenated lipophobic form of abscisic acid in the small intestine for inducing cellular apoptosis.

2. (canceled)

3. The medical solid composition as set forth in claim 1, further including a solid acceptable pharmaceutical carrier as a crystalline microcellulose i.p, at 53.9 W/W concentration.

4. The medical solid composition as set forth in claim 1, wherein the medical composition includes, for inducing cellular apoptosis, comprising in the medical solid component of the composition:
abscisic acid 10 mg
sodium bicarbonate 87.5 mg
carbonic acid 12.5 mg
methanol 0.2 ml as primary solvent of abscisic acid
tw 80 in powder formulation 2.5 ml
distilled water 4.2 ml in capillary form of water
microcrystalline cellulose as pharmaceutical carrier 134.8 mg

5. The medical solid composition as set forth in claim 1, wherein the medical composition includes, for inducing cell normalization, comprising the medical solid component of the composition:
abscisic acid 10 mg
sodium bicarbonate 50 mg
carbonic acid 50 mg
methanol 0.2 ml as primary solvent of abscisic acid
tw 80 in powder formulation 2.5 ml
distilled water 4.2 ml in capillary form of water
microcrystalline cellulose as pharmaceutical carrier 134.8 mg

6. The medical solid composition as set forth claim 1, further including a solid acceptable pharmaceutical carrier as a microtalc of Mg-Silicate with residues of magnesite, dolomite and chlorite, at 53.9% W/W concentration.

7. The medical solid composition as set forth in claim 1, further including a solid pharmaceutical carrier as a sodium starch glycolate at 53.9% W/W concentration.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)
